(19) United States
(12) Patent Application Publication
(10) Pub. No.: US 2024/0181023 A1
(43) Pub. Date: Jun. 6, 2024

(54) FUNCTIONALIZED BIOCATALYTICAL COMPOSITIONS

(71) Applicant: PERSEO PHARMA AG, Muttenz (CH)

(72) Inventors: Emilie LAPRÉVOTTE, Hegenheim (FR); Yves Victor René DUDAL, Bottmingen (CH); Manon BRIAND, Ennetbaden (CH); Emanuele CARLINI, München (DE); Patrick SHAHGALDIAN, Hegenheim (FR)

(21) Appl. No.: 18/556,287

(22) PCT Filed: Apr. 21, 2022
(86) PCT No.: PCT/EP2022/060562

§ 371 Date: Oct. 19, 2023

(30) Foreign Application Priority Data
Apr. 22, 2021 (EP) 21169848.5
Jan. 13, 2022 (EP) 22151420.1

(51) Int. Cl. A61K 38/50; A61K 9/51; A61P 35/00; C12N 11/14

(52) U.S. Cl. CPC A61K 38/50; A61K 9/5115; A61K 9/5169; A61K 9/5192; A61P 35/00; C12N 11/14; C12Y 305/01001

(57) ABSTRACT

The present invention relates to a composition comprising a solid carrier, an enzyme immobilized on the surface of the solid carrier, a protective layer to protect the enzyme by embedding the enzyme, and a functional constituent immobilized on the surface of the protective layer.

### THE FIELD OF THE INVENTION

The present invention relates to a composition comprising a solid carrier, an enzyme or a fragment thereof immobilized on the surface of the solid carrier, a protective layer to protect the enzyme or the fragment thereof by embedding the enzyme or the fragment thereof, and a functional constituent immobilized on the surface of the protective layer. The present invention also relates to methods of producing said composition.

### BACKGROUND OF THE INVENTION

Proteins such as enzymes are frequently needed, e.g. in industrial applications, diagnostics or for therapeutic use. In order to stabilize the proteins and/or to provide resistance to various types of stresses it has been suggested in the prior art to immobilize the proteins on the surface of a carrier and to protect them with a layer of protective material. Such an approach has been described e.g. in WO2015/014888 which discloses a biocatalytical composition comprising a solid carrier, an enzyme and a protective layer for protecting the enzyme by embedding the enzyme and a process to produce such biocatalytical composition. However biocatalytical compositions as described e.g in WO2015/014888 cannot be used in therapeutic application due to their lack of biocompability and bioavailability. Thus there is a need to provide biocatalytical compositions compatible and useful for therapeutic applications.

### SUMMARY OF THE INVENTION

The present invention provides a composition comprising a solid carrier, an enzyme or a fragment thereof immobilized on the surface of the solid carrier, a protective layer to protect the enzyme or the fragment thereof by embedding the enzyme or the fragment thereof, and a functional constituent immobilized on the surface of the protective layer.

The present invention provides also a method of producing said composition comprising a solid carrier, an enzyme or a fragment thereof immobilized on the surface of the solid carrier, a protective layer to protect the enzyme or the fragment thereof by embedding the enzyme or the fragment thereof, and a functional constituent immobilized on the surface of the protective layer, the method comprising the following steps:
(a) providing a solid carrier;
(b) immobilizing an enzyme or a fragment thereof on the solid carrier;
(c) forming a protective layer on the surface of the solid carrier to protect the enzyme or the fragment thereof immobilized on the solid carrier;
(d) immobilizing a functional constituent on the surface of the protective layer.

It has been surprisingly found by the inventors of the present application that compositions as provided by the present invention if applied therapeutically have an unexpected low anti-enzyme antibody response suggesting low or no immunogenicity, show low cytotoxicity, are not haemolytic and show fast clearance in the blood, thus making them extremely promising for therapeutic use.

### BRIEF DESCRIPTION OF THE FIGURES

FIG. 1) shows a schematic representation of the process for the production of the composition of the invention: a) an enzyme or fragment is immobilized on the solid carrier; b) and c) a protective layer grows around the immobilized enzyme or fragment thereof embedding the immobilized enzyme or fragment thereof, and d) a functional constituent is immobilized on the surface of the protective layer.

FIG. 2) shows functionalization of nanoparticles with PEG. Shielded nanoparticles (NP-1) were reacted with silane-PEG-FITC for 1 h, at 20° C. stirring at 400 rpm. Histogram represents the fluorescence intensity (lex: 489 nm; lem: 515 nm) of shielded- and reacted-nanoparticles.

FIG. 3) shows functionalization of nanoparticles with Human Serum Albumin (HSA). Shielded nanoparticles were incubated with HSA for 1 h, at 20° C. stirring at 400 rpm. Histogram represents the Lowry protein quantification of HSA in the supernatant of nanoparticles after reaction compared to initial solution of HSA.

FIG. 4) shows functionalization of nanoparticles with click chemistry Ethynyl-nanoparticles were incubated with N3-FITC for 6 h at 20° C. stirring at 400 rpm. Histogram represents the fluorescence polarization of free N3-FITC and N3-FITC immobilized on nanoparticles (lex: 489 nm; lem: 515 nm)

FIG. 5) shows sustained activity of NP-1 compared to free enzyme. Enzymatic activity was measured on NP-1 and compared to the equivalent amount of free enzyme over a period of 13 weeks. Data values are expressed as percentage of control at day 0.

FIG. 6) shows sustained activity of NP-1 in biological fluids. Enzymatic activity of NP-1 in biological fluids was assessed and compared to its activity in phosphate buffer. Activity data values are expressed as percentage of control in phosphate buffer.

FIG. 7) shows lower Ab-enzyme binding on NP-1. ELISA was assessed on nanoparticles at different stages of their development. ELISA data are expressed as enzyme concentration (mg/mL).

FIG. 8) shows resistance to proteolytic enzymes. NP-1, partially shielded- and free enzyme were exposed to proteases (10 mg/mL) for 20 h. Enzymatic activity data values are expressed as percentage of control at to.

FIG. 9) shows cytotoxicity assessment of NP-1 with LDH assay. HepG2 were exposed to increasing concentrations of enzyme-inactivated nanoparticles for 48 h. Cell damages were assessed using the lactate deshydrogenase (LDH) assay. Cytotoxicity data values of NP-1 are expressed as percentage of control cells treated with triton (1 mg/mL).

FIG. 10) shows hemolysis assessment of NP-1. Whole blood was incubated with increasing concentrations of NP-1 for 3 h at 37° C., mixing the samples every 30 min. (A) Photograph shows samples after centrifugation at 800 g for 15 min. (B) Histogram represents the percentage of hemolysis of nanoparticles treated samples compared to total blood hemoglobine: Triton (10 mg/mL) and PBS were used as positive and negative control, respectively.

FIG. 11) shows biodistribution of nanoparticles. NP-1-D (A-C) and NP-2-D (B-D) were injected intravenously in animals. Percent of ID recovered (% ID/g) in blood (A-B) in organs/tissues (C-D) were calculated at different terminal time points.

FIG. 12) shows the anti-tumor efficacy of NP-2. (A) PANC-1, MDA-MB-231 and JHH-5 cells were exposed to increasing concentrations of free asparaginase, NP-2, and inactivated NP-2 for 48 h. Cell viability was assessed using an MTT assay. Viability data is expressed as percentage of control cells treated with triton (1 mg/mL). (B) Enzymatic activity was measured on free asparaginase and NP-2 over 48 h. Activity was measured using Nessler's reaction.

FIG. 13) shows PBMCs proliferation. Freshly isolated PBMCs were labelled with CFSE and exposed to increasing concentrations of NP-1 and NP-2 (0 to 1000 ug/mL) or PHA (10 ug/mL) for 72 h. The histograms show the percentage of proliferating cells determined by flow cytometry.

FIG. 14) shows the anti-tumor efficacy of cancer-cells-targeting-nanoparticles. (A) RAJI cells were incubated with fluorescent NP-1 or fluorescent NP-5 for 30 min on ice. The specific binding of NP-1 and NP-5, respectively, on cancer cell was assessed by flow cytometry. The histogram represents the intensity of fluorescence of the cells. (B) RAJI cells were exposed to increasing concentration of free asparaginase, NP-1, NP-1 inactivated or NP-5 for 48 h. Cell viability was assessed using an MTT assay. Viability data is expressed as percentage of control cells treated with triton (1 mg/mL).

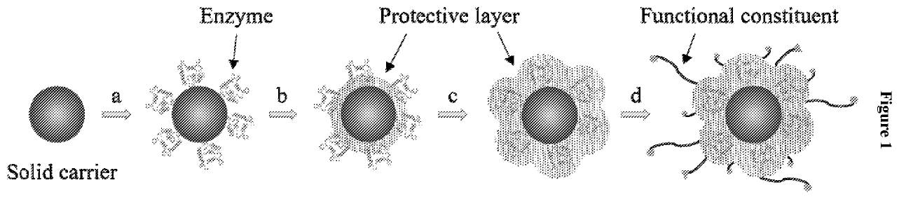

### DETAILED DESCRIPTION OF THE INVENTION

The present invention relates to a composition comprising a solid carrier, an enzyme or a fragment thereof immobilized on the surface of the solid carrier, a protective layer to protect the enzyme or the fragment thereof by embedding the enzyme or the fragment thereof, and a functional constituent immobilized on the surface of the protective layer.

For the purposes of interpreting this specification, the following definitions will apply and whenever appropriate, terms used in the singular will also include the plural and vice versa. It is to be understood that the terminology used herein is for the purpose of describing particular embodiments only and is not intended to be limiting.

Features, integers, characteristics, compounds described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments.

The term “comprise” and variations thereof, such as, “comprises” and “comprising” is generally used in the sense of include, that is, as “including, but not limited to”, that is to say permitting the presence of one or more features or components.

The singular forms “a,” “an,” and “the” include plural referents unless the content clearly dictates otherwise.

The term “about” refers to a range of values ±10% of a specified value. For example, the phrase “about 200” includes ±10% of 200, or from 180 to 220.

The term “solid carrier” as used herein refers usually to a particle. Preferably the solid carrier is a monodisperse particle or a polydisperse particle, more preferably a monodisperse particle. The solid carrier usually comprises organic particles, inorganic particles, organic-inorganic particles, self-assembling organic particles, silica particles, gold particles, titanium particles and is preferably a silica particle, more preferably a silica nanoparticle (SNP). The particle size of the solid carrier is usually between and 1 nm and 1000 μm, preferably between 10 nm and 100 μm, particularly about 50 nm.

The term “linker” or “cross-linker” which are used synonymously herein refers to any linking reagents containing groups, which are capable of binding to specific functional groups (e.g. primary amines, sulfhydryls, etc.). A linker in the context of the present invention usually connects the surface of the solid carrier with the enzyme. For example, a linker may be immobilized on the surface of the solid carrier e.g. on the silica surface as a carrier material and then the enzyme may be bound to an unoccupied binding-site of the linker. Alternatively, the linker may firstly bind to the enzyme and then the linker bound to the enzyme may bind with its unoccupied binding-site to the solid carrier. Various types of linkers are known in the art, including but not limited to straight or branched-chain carbon linkers, heterocyclic carbon linkers, peptide linkers, polyether linkers, and linkers that are known in the art as tags.

The term “protective layer” as used herein refers to a layer for protecting the functional properties of the enzyme immobilized on the surface of the solid carrier. The protective layer of the present invention is usually built with building blocks at least part of which are monomers capable of interacting with both each other usually by covalent binding and the immobilized enzyme usually by non-covalent binding. The protective layers are usually homogeneous layers where at least 50%, preferably at least 70%, more preferably at least 90% of the enzyme or fragment thereof are embedded in the protective layer.

The term, “enzyme or a fragment thereof” includes naturally occurring enzymes or a fragment thereof and also includes artificially engineered enzymes or a fragment thereof. Artificially engineered enzymes or a fragment thereof are e.g. variants or functionally active fragments of the enzyme. By “variants or functionally active fragments thereof” in relation to the enzyme of the present invention is meant that the fragment or variant (such as an analogue, derivative or mutant) is capable of exercising the same physiological function as the enzyme. Such variants include naturally occurring allelic variants and non-naturally occurring variants. Additions, deletions, substitutions and derivatizations of one or more of the amino acids are contemplated so long as the modifications do not result in loss of functional activity of the fragment or variant. Preferably the functionally active fragment or variant has at least about 80% sequence identity more preferably at least about 90% sequence identity, even more preferably at least about 95% sequence identity, most preferably at least about 98% sequence identity to the relevant part of the enzyme.

The term “partially embedded enzyme” as used herein shall mean that the enzyme is not fully covered by the protective layer, thus, the enzyme is not fully embedded in the protective layer. In one embodiment less than 50% of the enzyme of interest are covered by the protective layer, though typically more at least 70% will be covered, thus improving protection of the enzyme. In a preferred embodiment, at least 70%, more preferably at least 80%, even more preferably at least 90%, most preferably at least 95% of the enzyme of interest is covered by the protective layer. In another preferred embodiment, around 70% to around 95%, more preferrably around 80% to around 95%, even more preferably around 90% to around 95%, most preferably around 90% to around 95, 96, 97, 98 or 99% of the enzyme of interest are covered by the protective layer. In a particularly preferred embodiment, around 70%, particularly around 80%, more particularly around 90%, most particularly around 95% of the enzyme of interest is covered by the protective layer. In a more particularly preferred embodiment, around 70%, particularly around 80%, more particularly around 90%, most particularly around 95% of the enzyme of interest is covered by the protective layer, wherein the active site is not covered.

The term “fully embedded enzyme” as used herein shall mean that the enzyme of interest according to the invention is fully, i.e. 100% covered by the protective layer, i.e. that also the active site is covered.

The term “at least partially embedded enzyme” as used herein shall mean that the enzyme is at least partially embedded and may be fully embedded by the protective layer. Thus “at least partially embedded enzyme” means that the protective layer covers from about 30% and 100% of the enzyme or a fragment thereof, preferably from about 50% to about 100%, more preferably from about 80% to about 100%, even more preferably from about 90% to about 100%, most preferably from about 95% to about 100%, wherein the active site is preferably covered.

The term “functional constitutent” as used herein refers to a constituent which after being immobilized to the surface of the protective layer retains its characteristic, functional property. A functional constituent in the sense of the present invention can be e.g. an amphiphilic drug, an amino acid, a peptide, a protein or a fragment thereof, a silane copolymer or a combination of a protein or a fragment thereof and a silane copolymer, with the proviso that the protein or a fragment thereof is not the enzyme or a fragment thereof immobilized on the surface of the solid carrier.

The term “peptide” as used herein designates a series of amino acid residues connected to each other by peptide bonds between the alpha-amino and carboxy groups of adjacent residues and have usually an amino acid sequence comprising between at least 10 amino acids and not more than 100 amino acids.

The term “protein or fragment thereof” as used herein contains usually between 100 and 1500 amino acids, preferably between 100 and 800 amino acids, more preferably between 100 and 500 amino acids. A fragment of a protein as defined herein does usually have the same functional properties as the protein from which it is derived.

“Immunoglobulins” (Ig), also synonymously called “antibodies” herein, are generally comprising four polypeptide chains, two heavy (H) chains and two light (L) chains, and are therefore multimeric proteins, or an equivalent Ig homologue thereof (e.g., a camelid nanobody, which comprises only a heavy chain, single domain antibodies (dAbs) which can be either be derived from a heavy or light chain); including full length functional mutants, variants, or derivatives thereof (including, but not limited to, murine, chimeric, humanized and fully human antibodies, which retain the essential epitope binding features of an Ig molecule, and including dual specific, bispecific, multispecific, and dual variable domain immunoglobulins; Immunoglobulin molecules can be of any class (e.g., IgG, IgE, IgM, IgD, IgA, and IgY), or subclass (e.g., IgG1, IgG2, IgG3, IgG4, IgA1, and IgA2) and allotype. An “immunoglobulin fragment”, as used herein, relates to a molecule comprising at least one polypeptide chain derived from an antibody that is not full length, including, but not limited to (i) a Fab fragment, which is a monovalent fragment consisting of the variable light (VL), variable heavy (VH), constant light (CL) and constant heavy 1 (CHI) domains; (ii) a F(ab′)2 fragment, which is a bivalent fragment comprising two Fab fragments linked by a disulfide bridge at the hinge region; (iii) a heavy chain portion of a Fab(Fa) fragment, which consists of the VH and CHI domains; (iv) a variable fragment (Fv) fragment, which consists of the VL and VHdomains of a single arm of an antibody, (v) a domain antibody (dAb) fragment, which comprises a single variable domain; (vi) an isolated complementarity determining region (CDR); (vii) a single chain FvFragment (scFv); (viii) a diabody, which is a bivalent, bispecific antibody in which VH and VLdomains are expressed on a single polypeptide chain, but using a linker that is too short to allow for pairing between the two domains on the same chain, thereby forcing the domains to pair with the complementarity domains of another chain and creating two antigen binding sites; and (ix) a linear antibody, which comprises a pair of tandem Fvsegments (VH-CH1-VH-CH1) which, together with complementarity light chain polypeptides, form a pair of antigen binding regions; (x) a nanobody and (xi) other non-full length portions of immunoglobulin heavy and/or light chains, or mutants, variants, or derivatives thereof, alone or in any combination. A preferred immunoglobulin is an anti-CD19 antibody, in particular a human monoclonal anti-CD19 antibody.

In a first aspect the present invention provides a composition comprising a solid carrier, an enzyme or a fragment thereof immobilized on the surface of the solid carrier, a protective layer to protect the enzyme or the fragment thereof by embedding the enzyme or the fragment thereof, and a functional constituent immobilized on the surface of the protective layer.

The enzyme can be immobilized on the surface of the solid carrier by non-covalent binding or covalent binding. Non-covalent binding includes p-p (aromatic) interactions, van der Waals interactions, H-bonding interactions, and ionic interactions. Preferably the enzyme is immobilized on the surface of the solid carrier by covalent binding or by covalent binding via a linker.

In one embodiment the solid carrier is selected from the group of organic particles, inorganic particles, organic-inorganic particles, self-assembling organic particles, silica particles, gold particles, titanium particles and is preferably a silica particle, more preferably a silica nanoparticle (SNP). The particle size is usually measured by measuring the diameter of the particles and is usually between 1 nm and 1000 nm, preferably between 10 nm and 100 nm, particularly about 50 nm. In case the solid carrier is a monodisperse particle, the size is usually between 1 nm and 1000 nm, preferably between 10 nm and 100 nm, particularly about 50 nm. In case the solid carrier is a polydisperse particle, the size is usually between 1 nm and 1000 μm, preferably between 10 nm and 100 μm, particularly between 50 nm and 50 μm.

Usually monodisperse particles or polydisperse particles, preferably monodisperse particles are used as solid carrier in the present invention. In a preferred embodiment the monodisperse particles are spherical monodisperse particles. In a further preferred embodiment, the polydisperse particles are non-spherical polydisperse particles.

The solid carrier is usually provided in suspension. Suspension of the solid carrier can be e.g. in water, buffer or non-ionic surfactants or mixtures thereof, preferably in mixtures of water and non-ionic surfactants. Buffers which can be used in the method of the present invention are phosphate, piperazine-N,N′-bis(2-ethanesulfonic acid), 2-Hydroxy-3-morpholinopropanesulfonic acid, N,N-bis[2-hydroxyethyl]-2-aminoethanesulfonic acid), (3-(N-morpholino)propanesulfonic acid), 2-[[1,3-dihydroxy-2-(hydroxymethyl)propan-2-yl]amino]ethanesulfonic acid, 4-(2-hydroxyethyl)-1-piperazineethanesulfonic acid), 3-(N,N-Bis[2-hydroxyethyl]amino)-2-hydroxypropanesulfonic acid, N,N-Bis(2-hydroxyethyl)-3-amino-2-hydroxypropanesulfonic acid, N-[Tris(hydroxymethyl)methyl]glycine, Diglycine, 4-(2-Hydroxyethyl)-1-piperazinepropanesulfonic acid, N,N-Bis(2-hydroxyethyl)glycine, N-[Tris(hydroxymethyl)methyl]-3-aminopropanesulfonic acid, N-(1,1-Dimethyl-2-hydroxyethyl)-3-amino-2-hydroxypropanesulfonic acid.

In one embodiment the surface of the solid carrier is modified to introduce a molecule or functional chemical group as anchoring point i.e. as anchoring point for the enzyme or for the linker connecting the enzyme to the solid carrier. Preferably, said anchoring point is an amine functional chemical group or moiety. As a non-limiting example, an amino-modified surface of the solid carrier e.g. an amino-modified silica surface may be used as modified solid carrier. Such an amino-modified surface of the solid carrier may be obtained by reacting a solid carrier having a silica surface with an amino silane, e.g. with APTES. Thus in a preferred embodiment, the solid carrier is a solid carrier having a silica surface with an amino-modified surface, more preferably a solid carrier obtained by reacting the solid carrier having a silica surface with an amino silane, e.g. with APTES. Such a modified carrier may form an amide linkage between the enzyme and the amine group at the surface of the carrier material or an amide linkage between the linker and the amine group at the surface of the carrier material. In one embodiment the introduced molecule or functional chemical group as anchoring point is homogeneously distributed on the surface of the solid carrier. Preferably the surface of the solid carrier is only partially amino-modified. Thus in a preferred embodiment, the solid carrier is a solid carrier having a silica surface with an amino-modified surface, more preferably a solid carrier obtained by reacting the solid carrier having a silica surface with an amino silane, e.g. with APTES, even more preferably a solid carrier obtained by reacting the solid carrier having a silica surface partially with an amino silane, e.g. with APTES.

In some embodiments the protective layer has a defined thickness of about 1 to about 200 nm, usually 1 to about 100 nm, preferably about 1 to about 50 nm, more preferably about 1 to about 25 nm, even more preferably about 1 to about 20 nm, in particular about 1 to about 15 nm. The most preferred defined thickness is about 1 to about 15 nm. In some embodiments the layer has a defined thickness of about 5 to about 100 nm, preferably about 5 to about 50 nm, more preferably about 5 to about 25 nm, even more preferably about 5 to about 20 nm, in particular about 5 to about 15 nm. The most preferred defined thickness is about 5 to about 15 nm. The protective layer is usually porous and the pore size is between 1 and 100 nm, preferably between 1 and 20 nm.

In one embodiment, the enzyme or a fragment thereof is partially embedded by the protective layer. In another embodiment the enzyme or a fragment thereof is fully embedded by the protective layer. In a preferred embodiment the enzyme or a fragment thereof is at least partially embedded by the protective layer.

In one embodiment, the protective layer embeds the solid carrier and embeds the enzyme or a fragment thereof immobilized on the surface of the solid carrier. In one embodiment, the functional constituent immobilized on the surface of the protective layer is not embedded by the protective layer. Preferably, the protective layer fully embeds the solid carrier and fully embeds the enzyme or a fragment thereof immobilized on the surface of the solid carrier. More preferably, the protective layer fully embeds the solid carrier and fully embeds the enzyme or a fragment thereof immobilized on the surface of the solid carrier, wherein the functional constituent immobilized on the surface of the protective layer is not embedded by the protective layer. If the protective layer fully embeds the solid carrier and fully embeds the enzyme or a fragment thereof immobilized on the surface of the solid carrier, the enzyme is fully, i.e. 100% covered by the protective layer, so that also the active site is covered and the solid carrier is fully, i.e. 100% covered by the protective layer.

In one embodiment, the present invention comprises a composition comprising a solid carrier, an enzyme or a fragment thereof immobilized on the surface of the solid carrier, a protective layer to protect the enzyme or the fragment thereof by embedding the enzyme or the fragment thereof, and a functional constituent immobilized on the surface of the protective layer, wherein the functional constituent immobilized on the surface of the protective layer is different from the enzyme or the fragment thereof immobilized on the surface of the solid carrier. In one embodiment, the present invention comprises a composition comprising a solid carrier, an enzyme or a fragment thereof immobilized on the surface of the solid carrier, a protective layer to protect the enzyme or the fragment thereof by embedding the enzyme or the fragment thereof, and a functional constituent immobilized on the surface of the protective layer, wherein the functional constituent immobilized on the surface of the protective layer is not the enzyme or the fragment thereof immobilized on the surface of the solid carrier.

In one embodiment the enzyme or a fragment thereof is selected from the group consisting of oxidoreductases, transferases, hydrolases, lyases, isomerases, transpeptidases, or ligases, or a fragment thereof and mixtures thereof. Particular preferred is a hydrolase or a fragment thereof, more particular a hydrolase or a fragment thereof selected from the group consisting of a deaminase or a fragment thereof, a glucuronidase or a fragment thereof and a peptidase or a fragment thereof, even more particular a peptidase or a fragment thereof, preferably a peptidase or a fragment thereof selected from the group consisting of cysteinase, methioninase arginase and aspariginase, or a fragment thereof, most particular an aspariginase or a fragment thereof.

The protective layer thickness can be measured, by using a microscope such as scanning electron microscope (SEM), transmission electron microscopy (TEM), scanning probe microscopy (SPM), light scattering methods or by ellipsometry.

The composition of the present invention is usually produced in a reaction vessel like a reactor. The formation of the protective layer is usually carried out by forming the respective protective layer by building blocks, wherein the building blocks build the protective layer in a polycondensation reaction. The polycondensation can be effected in different solvents, preferably in aqueous solution. Polycondensation can be easily controlled and stopped if appropriate, allowing achievement of a defined thickness of the protective layer. The choice of the building blocks, which can be used to build the protective layer, may depend on the known structure of the enzyme in order to adapt the affinity of the protective layer according to optimal and/or desired parameters. As building blocks for the protective layer usually structural building blocks and protective building blocks are used to build the protective layer. Structural building blocks which can be used are e.g. tetraethylorthosilicate (designated herein as “TEOS” or “T”). Protective building blocks which can be used are e.g. 3-Aminopropyltriethoxysilane (designated herein as “APTES” or “A”), Propyltriethyoxysilane (designated herein as “PTES” or P”), Isobutyltriethoxysilane (designated as “IBTES”), Hydroxymethyltriethoxysilane (designated herein as “HTMEOS” or H), Benzyltriethoxysilane (designated herein as “BTES”), Ureidopropyltriethoxysilane (designated as “UPTES”), or Carboxyethyltriethoxysilane (designated herein as “CETES”). Structural building blocks are usually precursors of inorganic silica, capable of forming 4 covalent bonds in the layer formed. Protective building blocks are usually organosilanes, bearing an organic moiety endowed with the ability to interact with the enzymes (e.g., enzyme). Preferred structural building blocks are tetravalent silanes, in particular tetra-alkoxy-silanes. Preferred protective building blocks are trivalent silanes, in particular tri-alkoxy-silanes. More preferred structural building blocks are mixtures of tetravalent silanes and trivalent silanes, in particular mixtures of tetra-alkoxy-silanes and tri-alkoxy-silanes. Even more preferred structural building blocks are selected from the group consisting of tetraethylorthosilicate, tetra-(2-hydroxyethyl)silane, and tetramethylorthosilicate. Even more preferred protective building blocks are selected from the group consisting of carboxyethylsilanetriol, benzylsilanes, propylsilanes, isobutylsilanes, n-octylsilanes, hydroxysilanes, bis(2-hydroxyethyl)-3-aminopropylsilanes, aminopropylsilanes, ureidopropylsilanes, (N-Acetylglycyl)-3-aminopropylsilanes, hydroxy(polyethyleneoxy)propyl]triethoxysilanes, in particular selected from benzyltriethoxysilane, propyltriethoxysilane, isobutyltriethoxysilane, n-octyltriethoxysilane, hydroxymethyltriethoxysilane, bis(2-hydroxyethyl)-3-aminopropyltriethoxysilane, 3-Aminopropyltriethoxysilane, ureidopropyltriethoxysllane, (N-Acetylglycyl)-3-aminopropyltriethoxysilane, or selected from benzyltrimethoxysflane, propyltrimethoxysilane, isobutylimethoxysilane, n-octyltrimethoxysilane, hydroxyrnethyltrimethoxysilane, bis(2-hydroxyethyl)-3-aminopropyltrimethoxysilane, arninopropyltrimethoxysilane, ureidopropyltrimethoxysilane (N-Acetylglycyl)-3-aminopropyltrimethoxysilane or selected from benzyltrihydroxyethoxysilane, propyltrihydroxyethoxysilane, isobutyltrihydroxyethoxysilane, n-octyltrihydroxyethoxysilane, hydroxymefilyltrihydroxyethoxysilane, bis(2-hydroxyethyl)-3-aminopropyltrihydroxyethoxysilane, aminopropyltrihydroxyethoxysilane, Ureidopropyltrihydroxyethoxysilane (N-Acetylglycyl)-3-aminopropyltrihydroxymethoxysilane.

Particular preferred building blocks are TEOS as structural building block and APTES, PTES, and/or HTMEOS, preferably APTES as protective building block. In particular TEOS as structural building block and APTES as protective building block are used to build the protective layer.

The reaction time of the building blocks with the solid carrier depends on the length of the linker, if a linker is used, and the size of the enzyme. The reaction is usually carried out for a time period of between 0.5 to 10 hours, preferably between 1 and 5 hours, more preferably between 1 and 4 hours, even more preferably between 2 and 4 hours, preferably in aqueous solution and preferably at room temperature of about 5 to about 25° C. or at about 20° C. The formation of the protective layer can be stopped by actively stopping the polycondensation reaction e.g by removing the non-reacted building blocks e.g. by a washing step or by self-stopping of the polycondensation reaction caused by a limited amount of building blocks.

In a further more preferred embodiment the enzyme is immobilized on the solid carrier by at least partly modifying the surface of the solid carrier by introducing a molecule as anchoring point as described supra for the enzyme and by using a linker, preferably a cross-linker binding to the anchoring point and the enzyme.

In one embodiment the introduced molecule as anchoring point and/or the linker are homogeneously distributed on the surface of the solid carrier.

In a preferred embodiment the cross-linker is selected from the group consisting of glutaraldehyde, disuccinimidyl tartrate, bis[sulfosuccinimidyl]suberate, ethylene glycolbis(sulfosuccinimidylsuccinate), dimethyl adipimidate, dimethyl pimelimidate, sulfosuccinimidyl (4-iodoacetyl) aminobenzoate, 1,5-difluoro-2,4-dinitrobenzene, activated sulfhydrils, sulfhydryl-reactive 2-pyridyldithiol, BSOCOES (Bis[2-(succinimidooxycarbonyloxy)ethyl]sulfone), DSP (Dithiobis[succinimidyl]propionate]), DTSSP (3,3′-Dithiobis[sulfosuccinimidyl]propionate]), DTBP (Dimethyl 3,3′-dithiobispropionimidate 2 HCl), DST (Disuccinimidyl tartarate), Sulfo-LC-SMPT (4-Sulfosuccinimidyl-6-methyl-a-(2-pyridyldithio)toluamido]hexanoate)), SPDP (N-Succinimidyl 3-(2-pyridyldithio)-propionate), LC-SPDP (Succinimidyl 6-(3-[2-pyridyldithio]-propionamido)hexanoate), SMPT (4-Succinimidyloxycarbonyl-methyl-a-[2-pyridyldithio]toluene), DPDPB (1,4-Di-[3′-(2′-pyridyldithio)-propionamido]butane), DTME (Dithio-bismaleimidoethane), BMDB (1,4 bismaleimidyl-2,3-dihydroxybutane), Dibenzocyclooctyne-maleimide (DBCO-maleimide) and Azido PEG Maleimide (N3-PEG-maleimide). More preferably said cross-linker is selected from glutaraldehyde, disuccinimidyl tartrate, disuccinimidyl suberate, bis[sulfosuccinimidyl] suberate, ethylene glycolbis(sulfosuccinimidylsuccinate), dimethyl adipimidate, dimethyl pimelimidate, sulfosuccinimidyl (4-iodoacetyl) aminobenzoate, 1,5-difluoro-2,4-dinitrobenzene, activated sulfhydrils (e.g. suflhydryl-reactive 2-pyridyldithio), Dibenzocyclooctyne-maleimide (DBCO-maleimide) and Azido PEG Maleimide (N3-PEG-maleimide). Paticular preferred is glutaraldehyde, Dibenzocyclooctyne-maleimide (DBCO-maleimide) and Azido PEG Maleimide (N3-PEG-maleimide). Most preferred is glutaraldehyde. Some compounds of the above mentioned cross-linkers, like e.g. Dibenzocyclooctyne-maleimide (DBCO-maleimide) and Azido PEG Maleimide (N3-PEG-maleimide), respectively, form the cross linker by reacting with each other e.g. in situ e.g. in a “click chemistry” reaction (Copper-catalysed azide-alkyne cycloaddition, see e.g. Kolb et al. (2001) Angew. Chem. 40(11)2004-2021), e.g. Dibenzocyclooctyne-maleimide (DBCO-maleimide) can be bound to the surface of the protective layer and Azido PEG Maleimide (N3-PEG-maleimide) can be bound to the functionlisation constituent e.g. to an immunoglobulin, and the Dibenzocyclooctyne-maleimide (DBCO-maleimide) bound to the surface of the protective layer and the Azido PEG Maleimide (N3-PEG-maleimide) bound to the functionlisation constituent are reacted by click chemistry in situ to form a composition comprising a functional constituent immobilized on the surface of the protective layer via a cross-linker, wherein the cross-linker is composed of Dibenzocyclooctyne-maleimide (DBCO-maleimide) reacted with Azido PEG Maleimide (N3-PEG-maleimide). Thus in a preferred embodiment the cross-linker is composed of two compounds reacted by click chemistry, more preferably the cross-linker is composed of Dibenzocyclooctyne-maleimide (DBCO-maleimide) reacted with Azido PEG Maleimide (N3-PEG-maleimide).

After the protective layer has been formed, the solid carrier comprising the enzyme and the protective layer can be stored. Storing is usually accomplished e.g. by washing the composition formed e.g. with a buffer and storing it suspended or solved in that buffer for a desired time period. In a preferred embodiment the solid carrier comprising the enzyme and the protective layer is stored at a constant temperature between 2 to 25° C. In a further preferred embodiment, the solid carrier comprising the enzyme and the protective layer is stored 5 to 48 hours, preferably 10 to 30 hours. More preferably the solid carrier comprising the enzyme and the protective layer is stored at a constant temperature between 2 to 25° C., preferably at room temperature for 10 to 30 hours.

In one embodiment, the functional constituent i) reduces phagocytosis of the composition; ii) increases the circulation time of the composition, and/or iii) targets a tumor and/or promotes the internalization of the composition into tumor cells; when the composition of the present invention is administered to a subject, preferably the functional constituent reduces phagocytosis of the composition and/or increases the circulation time of the composition; or targets a tumor and/or promotes the internalization of the composition into tumor cells.

In one embodiment the functional constituent is selected from the group consisting of an amphiphilic drug, an amino acid, a peptide, a protein or a fragment thereof, a silane copolymer, and a combination of a protein or a fragment thereof and a silane copolymer, with the proviso that the protein or a fragment thereof is not the enzyme or a fragment thereof immobilized on the surface of the solid carrier. An amphiphilic drug is preferably a cationic amphiphilic drug. Characteristically, cationic amphiphilic drugs contain a hydrophobic part consisting of a nonpolar ring system and a hydrophilic group with one or more nitrogen groups which can bear a net positive charge at physiological pH. More preferably an amphiphilic drug is a cationic amphiphilic drug selected from the group consisting of Fluoxetine, Thirodazine, Promazine, Maprotiline, Loratadine, Imipramine, Doxepine, Desipramine, Clozapine, Clomipramine, Chlopromazine, Chloroquine, Labetalol, Dapoxetine, Fluvoxamine, Indalpine, Paroxetine, Zimelidine, Sertaline and Propanolol and salts, metabolites and prodrugs thereof. Further examples of suitable cationic amphiphilic drugs include the drugs stated above like Fluphenazine, Haloperidol (Haldol, Serenace), Prochlorperazine, Mesoridazine, Loxapine, Molindone (Moban), Perphenazine (Trilifon), Thiothixene (Navane), Trifluoperazine (Stelazine), Fluphenazine (Prolixin), Droperidol, Zuclopenthixol (Clopixol), Periciazine, Triflupromazine, Olanzapine, Quetiapine, Asenapine, Sulpiride, Amisulpiride, Remoxipride, Melperone, lloperidone, Paliperidone, Risperidone, Perospirone, Ziprasidone, Sertindole, Aripiprazole, Fluvoxamine (Luvox), Paroxetine (Paxil), Sertraline (Zoloft), Desvenlafaxine (Pristiq), Duloxetine (Cymbalta), Milnacipram (Ixel), Venlafaxine (Effexor), Mianserin (Tolvon), Mirtazapine, Atomoxetine (Strattera), Mazindol (Mazanor, Sanorex), Reboxetine (Edronax), Viloxazine (Vivalan), Bupropion, Tianeptine, Agomelatine, Amitriptyline (Elavil, Endep), Clomipramine (Anafranil), Doxepin (Adapin, Sinequan), Imipramine (Tofranil), Trimipramine (Surmontil), Nortriptyline (Pamelor, Aventyl), Protriptyline (Vivactil), Moclobemide (Aurorix, Manerix), Tranylcypromine (Parnate), Buspirone (Buspar), Gepirone (Ariza), Nefazodone (Serzone), Tandospirone (Sediel), Trazodone (Desyrel), Dosulepin, Etoperidone, Femoxetine, Lofepramine, Mazindol, Milnacipran, Nefazodone, Nisoxetine, Nomifensine, Oxaprotiline, Protryptiline, Viloxazine, Diphenhydramine, Loratadine, Desloratadine, Meclizine, Quetiapine, Fexofenadine Pheniramine, Cetirizine, Promethazine, Chlorpheniramine, Levocetirizine, Cimetidine, Famotidine, Ranitidine, Nizatidine, Roxatidine, Lafutidine, A-349,821, ABT-239, Ciproxifan Clobenpropit, Thioperamide, Thioperamide, JNJ 7777120, VUF-6002, Alprenolol, Bucindolol, Carteolol, Carvedilol, Labetalol, Nadolol, Penbutolol, Pindolol, Timolol, Acebutolol, Atenolol, Betaxolol, Bisoprolol, Celiprolol, Esmolol, Metoprolol, Nebivolol, and Butaxamine and salts, metabolites and prodrugs thereof. Even more preferably the amphiphilic drug is Chloroquine or Chlorpromazine. The amino acid is usually selected from the group consisting of alanine, arginine, asparagine aspartic acid, cysteine, glutamine, glutamic acid, glycine, histidine, isoleucine, leucine, lysine, methionine, phenylalanine, proline, serine, threonine, tryptophan, tyrosine, and valine, and is preferably lysine. The peptide is usually selected from the group consisting of cell-penetrating peptides like Penetratin (DOI: 10.1038/ncomms1952: Kondo E et al., Nat commun, 2012; Derossi D et al., J. Biol. Chem. 1994; 269:10444-10450) and RGD (tripeptide consisting of arginine, glycine and aspartate) and is preferably RGD. The protein or a fragment thereof is usually selected from the group consisting of serum albumin or a fragment thereof and an immunoglobulin or a fragment thereof and is preferably serum albumin or a fragment thereof, more preferably human serum albumin or a fragment thereof. The immunoglobulin or a fragment thereof is usually selected from the group consisting of a Fc or Fab fragment of an immunoglobulin, a Fc or Fab fragment of an immunoglobulin and a cross-linker, a monoclonal antibody and a nanobody and is preferably a Fc fragment of an immunoglobulin or a Fc fragment of an immunoglobulin and a cross-linker. The silane copolymer is usually selected from the group consisting of polyethylene glycol/silane copolymers, and polysorbate/silane copolymer and is preferably a silane PEG (PEG-Si), more preferably mSilane-PEG 2 kDa or a mSilane-PEG 5 kDa.

In a preferred embodiment the functional constituent is selected from the group consisting of serum albumin or a fragment thereof; serum albumin or a fragment thereof and a polyethylene glycol/silane copolymer; a polyethylene glycol/silane copolymer; a Fc fragment of an immunoglobulin; and a Fc fragment of an immunoglobulin and a cross-linker.

In a further preferred embodiment the functional constituent is selected from the group consisting of serum albumin or a fragment thereof, wherein the serum albumin or a fragment thereof binds to the surface of the protective layer; serum albumin or a fragment thereof and a polyethylene glycol/silane copolymer, wherein one part (end) of the polyethylene glycol/silane copolymer binds to the surface of the protective layer and the other part (end) to the serum albumin or the fragment thereof; a polyethylene glycol/silane copolymer wherein the polyethylene glycol/silane copolymer binds to the surface of the protective layer; a Fc or Fab fragment of an immunoglobulin; and a Fc or Fab fragment of an immunoglobulin and a cross-linker, wherein one part (end) of the cross-linker binds to the surface of the protective layer and the other part (end) to the Fc or Fab fragment of an immunoglobulin.

In a further preferred embodiment the functional constituent is a polyethylene glycol/silane copolymer, preferably a mSilane-PEG 2 kDa or a mSilane-PEG 5 kDa, wherein the polyethylene glycol/silane copolymer binds to the surface of the protective layer.

In a more preferred embodiment the functional constituent is selected from the group consisting of serum albumin or a fragment thereof; serum albumin or a fragment thereof and a polyethylene glycol/silane copolymer; a polyethylene glycol/silane copolymer; a Fc fragment of an immunoglobulin; and a Fc fragment of an immunoglobulin and a cross-linker, wherein the functional constituent reduces phagocytosis of the composition and/or increases the circulation time of the composition.

In a further more preferred embodiment the functional constituent is selected from the group consisting of serum albumin or a fragment thereof, preferably serum albumin or a fragment thereof, wherein the serum albumin or a fragment thereof binds to the surface of the protective layer; serum albumin or a fragment thereof and a polyethylene glycol/silane copolymer, wherein one part (end) of the polyethylene glycol/silane copolymer binds to the surface of the protective layer and the other part (end) to the serum albumin or the fragment thereof; a polyethylene glycol/silane copolymer wherein the polyethylene glycol/silane copolymer binds to the surface of the protective layer; a Fc or Fab fragment of an immunoglobulin; and a Fc or Fab fragment of an immunoglobulin and a cross-linker, wherein one part (end) of the cross-linker binds to the surface of the protective layer and the other part (end) to the Fc or Fab fragment of an immunoglobulin, wherein the functional constituent reduces phagocytosis of the composition and/or increases the circulation time of the composition.

In an even more preferred embodiment the functional constituent is serum albumin or a fragment thereof, preferably serum albumin. Preferably, the serum albumin or a fragment thereof binds to the surface of the protective layer. The serum albumin or a fragment thereof used as functional constituent herein is preferably human and/or recombinant serum albumin or a fragment thereof, more preferably human serum albumin or a fragment thereof.

In a further even more preferred embodiment the functional constituent is serum albumin or a fragment thereof, preferably serum albumin, wherein the functional constituent reduces phagocytosis of the composition and/or increases the circulation time of the composition.

In a further even more preferred embodiment the functional constituent is a polyethylene glycol/silane copolymer, preferably a mSilane-PEG 2 kDa or a mSilane-PEG 5 kDa, wherein the polyethylene glycol/silane copolymer binds to the surface of the protective layer.

In a further even more preferred embodiment the functional constituent is a polyethylene glycol/silane copolymer, preferably a mSilane-PEG 2 kDa or a mSilane-PEG 5 kDa, wherein the polyethylene glycol/silane copolymer binds to the surface of the protective layer, wherein the functional constituent reduces phagocytosis of the composition and/or increases the circulation time of the composition.

In one embodiment the functional constituent is selected from the group consisting of a peptide; a peptide and a cross-linker; an immunoglobulin or a fragment thereof; and an immunoglobulin or a fragment thereof and a cross-linker.

In a preferred embodiment the functional constituent is selected from the group consisting of a peptide wherein the peptide binds to the surface of the protective layer; a peptide and a cross-linker wherein one part (end) of the cross-linker binds to the surface of the protective layer and the other part (end) to the peptide; an immunoglobulin or a fragment thereof wherein the immunoglobulin or a fragment thereof binds to the surface of the protective layer; and an immunoglobulin or a fragment thereof and a cross-linker wherein one part (end) of the cross-linker binds to the surface of the protective layer and the other part (end) to the immunoglobulin or a fragment thereof.

In a more preferred embodiment the functional constituent is selected from the group consisting of a peptide wherein the peptide binds to the surface of the protective layer; a peptide and a cross-linker wherein one part (end) of the cross-linker binds to the surface of the protective layer and the other part (end) to the peptide; an immunoglobulin or a fragment thereof wherein the immunoglobulin or a fragment thereof binds to the surface of the protective layer; and an immunoglobulin or a fragment thereof and a cross-linker wherein one part (end) of the cross-linker binds to the surface of the protective layer and the other part (end) to the immunoglobulin or a fragment thereof, wherein the peptide and the immunoglobulin or a fragment thereof targets a tumor and/or promotes the internalization of the composition into tumor cells.

In a preferred embodiment the functional constituent is selected from the group consisting of a protein or a fragment thereof, preferably serum albumin or a fragment thereof; a silane copolymer, preferably a polyethylene glycol/silane copolymer; an immunoglobulin or a fragment thereof, preferably an antibody or a fragment thereof; and an immunoglobulin or a fragment thereof and a cross-linker, preferably an antibody or a fragment thereof and a cross-linker, preferably an antibody or a fragment thereof and a cross-linker composed of two compounds reacted by click chemistry.

In a preferred embodiment the functional constituent is selected from the group consisting of a protein or a fragment thereof, preferably serum albumin or a fragment thereof; a silane copolymer, preferably a polyethylene glycol/silane copolymer; an immunoglobulin or a fragment thereof, preferably an antibody or a fragment thereof; and an immunoglobulin or a fragment thereof and a cross-linker, preferably an antibody or a fragment thereof and a cross-linker, preferably an antibody or a fragment thereof and a cross-linker composed of two compounds reacted by click chemistry, wherein the functional constituent reduces phagocytosis of the composition and/or increases the circulation time of the composition and/or targets a tumor and/or promotes the internalization of the composition into tumor cells, preferably wherein the functional constituent increases the circulation time of the composition and targets a tumor.

In one embodiment the surface of the protective layer is only partially covered by the immobilized functional constituent. Preferably, between about 0.1% and about 100% of the surface of the protective layer are covered by the immobilized functional constituent. More preferably between about 5% and about 80%, even more preferably between about 10% and about 50%, most preferably about 20% of the surface of the protective layer are covered by the immobilized functional constituent.

In one embodiment the functional constituent is immobilized on the surface of the protective layer by binding, preferably covalent binding.

The immobilization of the functional constituent to the surface of the protective layer is usually carried in a reaction vessel like a reactor by suspending the solid carrier carrying the enzyme embedded in a protective layer as described supra in e.g. in water, buffer or non-ionic surfactants or mixtures thereof, preferably in mixtures of water and non-ionic surfactants. The functional component is then added to the suspension to react usually under stirring with the surface of the protective layer to immobilize the functional constitutent on the surface of the protective layer. Usually such obtained composition is washed and resuspended into water, buffer or non-ionic surfactants or mixtures thereof. Depending on the functional constituent used immobilization takes place by polycondensation e.g. in case a PEG silane is used as functional constituent or by covalent binding e.g. in case a protein is used as functional constituent. The functional constituent may also be immobilized by chemically modifying the surface of the protective layer and the functional constituent using e.g. “click chemistry” (Copper-catalysed azide-alkyne cycloaddition, see e.g. Kolb et al. (2001) Angew. Chem. 40(11)2004-2021), whereas the solid carrier carrying the enzyme embedded in a protective layer as described supra is first reacted with a reactive compound like an ethynyl compound and the functional constituent is modified by adding a reactive compound e.g. an azide residue and then both components are reacted to immobilize the functional constituent on the surface of the protective layer.

In a further embodiment the composition further comprises a chelating agent, wherein the chelating agent optionally comprises a radioactive or luminescent label. Preferably a chelating agent is selected from the group consisting of DOTA, DTPA, NOTA, TETA, AAZTA, TRAP, NOPO and HEHA. More preferably DOTA or HEHA are used. Even more preferably a chelating agent which comprises a radioactive or luminescent label is used, in particular p-SCN-Bn-DOTA or Lutetium-177-radiolabeled-DOTA is used. If the composition further comprises a chelating agent, the solid carrier carrying the enzyme embedded in a protective layer is usually pretreated with a chelating agent and a different chelating agent which comprises a radioactive or luminescent label is added to the such pretreated composition. Preferably a radioactive label is used, more preferably a compound of the lanthanides family, even more preferably Gadolinium, Lutetium, or Europium.

In a further aspect the present invention provides the composition as described supra for use as a medicament.

In a further aspect the present invention provides the composition for use in a method for the prevention, delay of progression or treatment cancer in a subject, the method comprising administering to the subject said composition, wherein the composition is administered in an amount that is sufficient to treat the subject. Also provided is the use of the composition as described herein for the manufacture of a medicament for the prevention, delay of progression or treatment of cancer in a subject. Also provided is the use of the composition as described herein for the prevention, delay of progression or treatment of cancer in a subject. Also provided is a method for the prevention, delay of progression or treatment of cancer in a subject, comprising administering to said subject a therapeutically effective amount of the composition as described herein. In the method for the prevention, delay of progression or treatment cancer in a subject, the functional constituent immobilized on the surface of the protective layer in the composition of the invention is preferably selected from the group consisting of an amphiphilic drug, an amino acid, a peptide, a protein or a fragment thereof, a silane copolymer, and a combination of a protein or a fragment thereof and a silane copolymer, with the proviso that the protein or the fragment thereof is not the enzyme or the fragment thereof immobilized on the surface of the solid carrier; more preferably selected from the group consisting of serum albumin or a fragment thereof, wherein the serum albumin or a fragment thereof binds to the surface of the protective layer; serum albumin or a fragment thereof and a polyethylene glycol/silane copolymer, wherein one part (end) of the polyethylene glycol/silane copolymer binds to the surface of the protective layer and the other part (end) to the serum albumin or the fragment thereof; a polyethylene glycol/silane copolymer wherein the polyethylene glycol/silane copolymer binds to the surface of the protective layer; a Fc or Fab fragment of an immunoglobulin; and a Fc or Fab fragment of an immunoglobulin and a cross-linker, wherein one part (end) of the cross-linker binds to the surface of the protective layer and the other part (end) to the Fc or Fab fragment of an immunoglobulin; and an even more preferably serum albumin or a fragment thereof or a polyethylene glycol/silane copolymer, in particular serum albumin or a mSilane-PEG 2 kDa or a mSilane-PEG 5 kDa, more particular human and/or recombinant serum albumin or a fragment thereof or a mSilane-PEG 2 kDa or a mSilane-PEG 5 kDa, even more particular human serum albumin or a fragment thereof or a mSilane-PEG 2 kDa or a mSilane-PEG 5 kDa, even more particular a protein or a fragment thereof, preferably serum albumin or a fragment thereof; a silane copolymer, preferably a polyethylene glycol/silane copolymer; an immunoglobulin or a fragment thereof, preferably an antibody or a fragment thereof, and an immunoglobulin or a fragment thereof and a cross-linker, preferably an antibody or a fragment thereof and a cross-linker, preferably an antibody or a fragment thereof and a cross-linker composed of two compounds reacted by click chemistry.

The terms “treatment”/“treating” as used herein includes: (1) delaying the appearance of clinical symptoms of the state, disorder or condition developing in an animal, particularly a mammal and especially a human, that may be afflicted with or predisposed to the state, disorder or condition but does not yet experience or display clinical or subclinical symptoms of the state, disorder or condition; (2) inhibiting the state, disorder or condition (e.g. arresting, reducing or delaying the development of the disease, or a relapse thereof in case of maintenance treatment, of at least one clinical or subclinical symptom thereof); and/or (3) relieving the condition (i.e. causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be effective treatment.

As used herein, “delay of progression” means increasing the time to appearance of a symptom of a cancer or a mark associated with a cancer or slowing the increase in severity of a symptom of a cancer. Further, “delay of progression” as used herein includes reversing or inhibition of disease progression. “Inhibition” of disease progression or disease complication in a subject means preventing or reducing the disease progression and/or disease complication in the subject.

Preventive treatments comprise prophylactic treatments. In preventive applications, the pharmaceutical combination of the invention is administered to a subject suspected of having, or at risk for developing cancer. In therapeutic applications, the pharmaceutical combination is administered to a subject such as a patient already suffering from cancer, in an amount sufficient to cure or at least partially arrest the symptoms of the disease. Amounts effective for this use will depend on the severity and course of the disease, previous therapy, the subject's health status and response to the drugs, and the judgment of the treating physician. In the case wherein the subject's condition does not improve, the pharmaceutical combination of the invention may be administered chronically, which is, for an extended period of time, including throughout the duration of the subject's life in order to ameliorate or otherwise control or limit the symptoms of the subject's disease or condition.

In the case wherein the subject's status does improve, the pharmaceutical combination may be administered continuously; alternatively, the dose of drugs being administered may be temporarily reduced or temporarily suspended for a certain length of time (i.e., a “drug holiday”). Once improvement of the patient's condition has occurred, a maintenance dose of the pharmaceutical combination of the invention is administered if necessary. Subsequently, the dosage or the frequency of administration, or both, is optionally reduced, as a function of the symptoms, to a level at which the improved disease is retained.

The expression “effective amount” or “therapeutically effective amount” as used herein refers to an amount capable of invoking one or more of the following effects in a subject receiving the combination of the present invention: (i) inhibition or arrest of tumor growth, including, reducing the rate of tumor growth or causing complete growth arrest; (ii) complete tumour regression; (iii) reduction in tumor size; (iv) reduction in tumor number; (v) inhibition of metastasis (i.e., reduction, slowing down or complete stopping) of tumor cell infiltration into peripheral organs; (vi) enhancement of antitumor immune response, which may, but does not have to, result in the regression or elimination of the tumor; (vii) relief, to some extent, of one or more symptoms associated with cancer; (viii) increase in progression-free survival (PFS) and/or; overall survival (OS) of the subject receiving the combination.

Determination of a therapeutically effective amount is well within the capability of those skilled in the art, especially in light of the detailed disclosure provided herein. In some embodiments, a therapeutically effective amount may (i) reduce the number of cancer cells; (ii) reduce tumor size; (iii) inhibit, retard, slow to some extent, and preferably stop cancer cell infiltration into peripheral organs; (iv) inhibit (e.g., slow to some extent and preferably stop) tumor metastasis; (v) inhibit tumor growth; (vi) delay occurrence and/or recurrence of a tumor; and/or (vii) relieve to some extent one or more of the symptoms associated with the cancer. In various embodiments, the amount is sufficient to ameliorate, palliate, lessen, and/or delay one or more of symptoms of cancer.

In one embodiment the cancer is a solid tumor. In a further embodiment the cancer is a haematological malignancy. In a preferred embodiment the cancer is leukemia. In a more preferred embodiment the cancer is a lymphoblastoid cancer, more preferably a lymphoblastoid cancer selected from the group consisting of Burkitt lymphoma or T-cell acute lymphoblastic leukemia (T-ALL).

In a further aspect the present invention provides the composition as described supra for use in a method for measuring the distribution of the composition in a subject. In one embodiment, the method for measuring the distribution of the composition in a subject comprises administering the composition comprising a solid carrier, an enzyme or a fragment thereof immobilized on the surface of the solid carrier, a protective layer to protect the enzyme or the fragment thereof by embedding the enzyme or the fragment thereof, and a functional constituent immobilized on the surface of the protective layer, and further comprising a chelating agent, wherein the chelating agent comprises a radioactive or luminescent label, to the subject, and analyzing the radioactive or luminescent emission.

The present invention provides also a method to measure the distribution of the composition in a subject, comprising administering the composition comprising a solid carrier, an enzyme or a fragment thereof immobilized on the surface of the solid carrier, a protective layer to protect the enzyme or the fragment thereof by embedding the enzyme or the fragment thereof, and a functional constituent immobilized on the surface of the protective layer, and further comprising a chelating agent, wherein the chelating agent comprises a radioactive or luminescent label, to the subject, and analyzing the radioactive or luminescent emission.

In a further aspect the present invention provides a method of producing a composition as described supra, e.g. a composition a solid carrier, an enzyme or a fragment thereof immobilized on the surface of the solid carrier, a protective layer to protect the enzyme or the fragment thereof by embedding the enzyme or the fragment thereof, and a functional constituent immobilized on the surface of the protective layer.; wherein the method comprises the following steps:
(a) providing a solid carrier;
(b) immobilizing an enzyme or a fragment thereof on the solid carrier;
(c) forming a protective layer on the surface of the solid carrier to protect the enzyme or the fragment thereof immobilized on the solid carrier;
(d) immobilizing a functional constituent on the surface of the protective layer.

Step (a) is usually carried out by providing the solid carrier in suspension in water or a buffer. The suspension can be stirred e.g at 400 rpm, 20° C. for 30 min. The immobilization of the enzyme on the solid carrier in step b) of the present method is usually carried out by adding a solution of the enzyme to the suspension of the solid carrier. In a preferred embodiment the immobilization of the enzyme on the solid carrier is carried out by providing a suspension of the solid carrier and adding a solution of the enzyme, wherein the suspension with the added solution of the enzyme is incubated to allow the enzyme to bind on the surface of the solid carrier. In a preferred embodiment the surface of the solid carrier is at least partly modified to improve immobilization of the enzyme on the solid carrier. In particular, the surface of the solid carrier is at least partly modified before the enzyme is immobilized. The surface of the solid carrier can be at least partly modified by introducing a molecule as anchoring point for the enzyme to the surface of the solid carrier as described supra. The formation of the protective layer according to step (c) of the present method is usually carried out by forming the respective protective layer with building blocks, wherein the building blocks build the protective layer in a polycondensation reaction as described supa. The immobilization of a functional constituent on the surface of the protective layer according to step (d) of the present method is usually carried out as described supra.

In one embodiment the protective layer is formed by building blocks, wherein as building blocks structural building blocks and protective building blocks are used to form the protective layer, wherein the structural building blocks are precursors of inorganic silica, capable of forming 4 covalent bonds in the layer formed and the protective building blocks are organosilanes.

In one embodiment the protective layer embeds from about 30% to about 100% of the enzyme.

In one embodiment the solid carrier is selected from the group of organic particles, inorganic particles, organic-inorganic particles, self-assembled organic particles, silica particles, gold particles, magnetic particles and titanium particles.

### EXAMPLES

### Material and Methods:

### Reagents:

Tetraethyl orthosilicate 99% (TEOS), (3-aminopropyl)-triethoxysilane (APTES), ammonium hydroxide (ACS grade, 28-30%), ethanol (ACS grade, anhydrous), glutaraldehyde (grade I, 25% in water), copper sulfate, sodium ascorbate, Chelex® 100 sodium form, asparaginase (EC3.5.1.1), L-asparagine, trichloroacetic acid, Nessler's reagent, ethanolamine, tween 20, hemoglobin standard, Drabkin's solution, Triton X, digitonin, and Dibenzocyclooctyne-maleimide (DBCO-maleimide) were purchased from Sigma-Aldrich. p-SCN-Bn-DOTA was purchased from Macrocyclics.

Triethoxyethynylsilane (ETES) was purchased from Toronto Research Chemicals.

Methoxy PEG silane with various molecular weight (PEG-silane), Silane-PEG-fluorescein isothiocyanate (Silane-PEG-FITC) and Azido PEG Maleimide (N3-PEG-maleimide) were purchased from Nanocs.

Human Serum Albumin (HSA) was purchased from Fitzgerald.

Polyclonal anti-asparaginase antibody (LS-C147330) was purchased from LS-Bio and HRP-conjugated goat anti-rabbit antibody (43R-IG036hrp) from Fitzgerald. Amine-reactive 96-well plates (bearing NHS group) were purchased from Interchim. HRP colorimetric substrate 3,3,5,5-tetramethylbenzidine (TMB) and Dulbecco's phosphate-buffered saline (DPBS) were purchased from Thermo Scientific.

LDH activity assay kit was purchase at Biovision.

Dimethyl sulfoxide (DMSO), MTT ((3-[4,5-dimethylthiazol-2-yl]-2,5 diphenyl tetrazolium bromide), PHA (phytohemagglutinin) were purchased from Sigma.

RAJI, PANC-1 and MDA-MB-231 cells were purchased from LGC.

JHH5 cells were purchased from TebuBio.

CFSE (carboxyfluorescein diacetate, succinimidyl ester) was purchased from Thermo Fischer Scientific.

Buffy coats were obtained from the Basel blood donation center (Blutspendezentrum SRK Beider Basel).

Human CD19 Antibody (anti-CD19 antibody) was purchased from Bio-Techne.

Sodium carbonate (≥99.5%, Ph. Eur., USP, BP, anhydrous) and Sodium hydrogen carbonate (≥99.5%, CELLPURE©) were purchased from Carl Roth.

### Synthesis Silica Nanoparticles:

Silica nanoparticles of 50 nm particle size have been synthetized following the original Stöber process as described in WO2015/014888 A1. Briefly, ethanol, distilled water (6M) and ammonium hydroxide (0.13M) were mixed and stirred at 400 rpm for 1 h. TEOS (0.28M) was added and the solution was stirred at 400 rpm at 20° C. for 22 h. The solution was then centrifuged at 20 000 g for 20 min and washed successively with ethanol and water. Particle size measurement was carried out on SEM micrographs acquired at a magnification of 150 000× using the image analysis software Olympus stream motion.

### Enzyme Shielding:

Silica nanoparticles in water-polysorbate 80 (8 mg/L) were reacted with APTES (2.75 mM) for 30 min at 20° C. under stirring (400 rpm). Unreacted reagents were removed from the nanoparticles suspension using the amicon stirred cells with 300 kDa NMWL, Biomax polyethersulfone ultrafiltration discs, and the nanoparticles suspension were sonicated at 62.5 Watts for 5 min on ice (hereafter called “washing step”). Nanoparticles were then incubated with 0.1% (v/v) of aqueous glutaraldehyde solution for 30 min at 20° C. under stirring (400 rpm). After a washing step, the nanoparticles were resuspended in MES buffer (10 mM, pH 6.2) with polysorbate 80 (8 mg/L) and reacted with asparaginase (20 μg/mL) for 1 h at 20° C. under stirring (400 rpm). The nanoparticles were washed and TEOS (42.5 mM) was added and allowed to react for 1 h at 20° C. under stirring (400 rpm). Subsequently, APTES (4.5 mM) was added to the reaction mixture. The silane polycondensation was stopped after 21 h by washing the nanoparticles suspension. The silica nanoparticles obtained after silane polycondensation comprise the enzyme asparaginase immobilized on the surface of the silica particle which is fully embedded by the protective layer comprising polycondensed silanes. These nanoparticles which have been produced as described in WO2015/014888 A1 are further referred herein as “shielded nanoparticles”, “enzyme shielded nanoparticles”, “Nanoparticles 1” or “NP-1”. Particle size measurement was carried out on SEM micrographs acquired at a magnification of 150 000× using the image analysis software Olympus stream motion.

### Inactivation of Nanoparticles

Enzyme shielded nanoparticles produced according to section “Enzyme shielding” above were thermally inactivated by heating the nanoparticles at 95° C. for 20 min and are referred herein below as “enzyme-inactivated-NP” or “inactivated-NP”.

### Functionalization of Nanoparticles:

Functionalization with PEG:

Enzyme shielded nanoparticles produced according to section “Enzyme shielding” above in MES buffer (10 mM, pH 6,2) with polysorbate 80 (8 mg/L), were reacted with PEG silane 5000 or PEG-Silane-FITC (1 mg/mL) for 1 h at 20° C. under stirring (400 rpm). After a washing step, the nanoparticles were resuspended in MES buffer (10 mM, pH 6.2) with polysorbate 80 (8 mg/L). The silica nanoparticles obtained after functionalization with PEG comprise the enzyme asparaginase immobilized on the surface of the silica particle which is fully embedded by the protective layer comprising polycondensed silanes and further comprises PEG silane as functional constituent immobilized on the surface of the protective layer. These nanoparticles functionalized with PEG silane 5000 are further referred herein as “Nanoparticles 2” or “NP-2”.

Functionalization with Human Serum Albumin:

Enzyme shielded nanoparticles produced according to section “Enzyme shielding” above in MES buffer (10 mM, pH 6.2) were reacted with HSA (20 μg/mL) for 1 h at 20° C. under stirring (400 rpm). After a washing step, the nanoparticles were resuspended in MES buffer (10 mM, pH 6.2) with polysorbate 80 (8 mg/L). The silica nanoparticles obtained after functionalization with PEG comprise the enzyme asparaginase immobilized on the surface of the silica particle which is fully embedded by the protective layer comprising polycondensed silanes and further comprises HSA as functional constituent immobilized on the surface of the protective layer. These nanoparticles functionalized with HSA are further referred herein as “Nanoparticles 3” or “NP-3”.

### Functionalization Using Click Chemistry:

Enzyme shielded nanoparticles produced according to section “Enzyme shielding” above in Borate buffer (50 mM, pH 8,5) with polysorbate 80 (8 mg/L) were reacted with ETES (80 mM) for 1 h at 20° C. under stirring (400 rpm). After a washing step, ethynyl-nanoparticles were resuspended in MES buffer (10 mM, pH 6.2), and N₃ modified compounds were added (40 μM) in presence of sodium ascorbate (4 μM) and copper sulfate (0.4 μM) and allowed to react for 6 h at 20° C. under stirring (400 rpm). After a washing step, the nanoparticles were resuspended in MES buffer (10 mM, pH 6.2) with polysorbate 80 (8 mg/L). The silica nanoparticles obtained after functionalization with PEG comprise the enzyme asparaginase immobilized on the surface of the silica particle and fully embedded by the protective layer of polycondensed silanes and further comprises N3 modified compounds such as N3-FITC as functional constituent immobilized on the surface of the protective layer. These nanoparticles functionalized with N3 modified compounds are further referred herein as “Nanoparticles 4” or “NP-4”.

Functionalization with Antibodies

To anti-CD19 antibodies (64 μL, 1 mg/mL) in DPBS buffer (10 mM, pH 7.4) was added 26 μL of carbonate buffer (50 mM, pH 9.2). Then, N3-PEG-maleimide (2.56 μL, 100 μg/mL) in sterile water was added to the anti-CD19 antibodies solution and reacted for 1 h at 20° C. under stirring (400 rpm) to yield azido anti-CD19 antibodies.

Enzyme shielded nanoparticles (600 μL, 10 mg/mL) produced according to section “Enzyme shielding” above in MES buffer (10 mM, pH 6,2) with polysorbate 80 (8 mg/L), were resuspended in carbonate buffer (50 mM, pH 9.2) and subsequently reacted with DBCO-maleimide (1.82 μL, 100 μg/mL) for 1 h at 20° C. under stirring (400 rpm) to yield DBCO enzyme shielded nanoparticles. After a washing step, the DBCO enzyme shielded nanoparticles were resuspended in DPBS buffer (10 mM, pH 7.4) and reacted with azido anti-CD19 antibodies for 5 h at 20° C. under stirring (400 rpm). After a washing step, the nanoparticles were resuspended in DPBS buffer (10 mM, pH 7.4). The silica nanoparticles obtained after functionalization with anti-CD19 antibody comprise the enzyme asparaginase immobilized on the surface of the silica nanoparticle which is fully embedded by the protective layer comprising polycondensed silanes and further comprises anti-CD19 antibody as functional constituent immobilized on the surface of the protective layer. These nanoparticles functionalized with anti-CD19 antibody are further referred herein as “Nanoparticles 5” or “NP-5”.

### Labeling of Nanoparticles:

Enzyme shielded nanoparticles produced according to section “Enzyme shielding” above were resuspended in phosphate buffer (0.1M, pH 7.4) with polysorbate 80 (8 mg/L), pretreated with Chelex®, and p-SCN-Bn-DOTA (1 mg/mL) was added and allowed to react for 1 h at 20° C. under stirring (400 rpm). After a washing step, the nanoparticles were resuspended in MES buffer (10 mM, pH 6.2) with polysorbate 80 (8 mg/L). These particles are incubated with 2800 μCi of 177-Lutetium for 12 hours at 45° C., and further washed prior to injection.

### Protein Quantification:

Immobilization yield of HSA was quantified using the indirect Lowry protein quantification method. A standard regression curve with known concentrations of protein was build using Bovine Serum Albumin standards. The supernatant of enzyme shielded nanoparticles after HSA immobilization (HSA immobilization was performed according to section “Functionalization with Human Serum Albumin” above) was taken and centrifuged for 3 minutes at 20 k rcf. Then, 1 mL of Lowry solution was added to 200 μL of samples and standards, vortexed and incubated at room temperature for 15 minutes. Subsequently, 100 μL of Folin reagent 1N were added while vortexing and incubated for 30 minutes at room temperature. Finally, the absorbance was read at 750 nm using Biotek Synergy H1 Reader.

### Asparaginase Activity Assay:

Free asparaginase or shielded nanoparticle-asparaginase (0.1 to 1 μg/mL) were mixed with 10 mM L-asparagine in phosphate buffer (0.5M, pH 7.4), albumin (40 mg/mL) or whole blood to a final volume of 200 μL and incubated for 30 min at 37° C. for the enzymatic reaction. The reaction was stopped with 50 μl of 1.5M trichloroacetic acid and centrifuged. Two-hundred microliters of mixture supernatant were mixed with equal volume of distilled water before the addition of 100 μL of Nessler's reagent. The absorbance was measured at 436 nm. Enzyme activity was quantified based on the standard curve of ammonia obtained by Nessler's reagent.

### ELISA Procedure:

Wells of amine-reactive plates were coated with 0 to 50 μg/ml of shielded nanoparticle-asparaginase or free asparaginase in 100 μl of PBS for 2 h at room temperature (RT). Then NHS functions was quenched using ethanolamine (100 mM, pH 7) for 30 min at RT and wells were blocked with 5% Milk in PBS for 1 h at RT. After incubation with anti-asparaginase antibody (1 μg/mL) in antibody dilution buffer (PBS-Milk 1%, 0.1% (v/v) Tween 20) for 2 h at RT, HRP-conjugated goat anti-rabbit IgG (1:10,000) in antibody dilution buffer was added for 1 h at RT. Finally, the reaction was visualized by the addition of 50 μl (TMB) for 30 min. The reaction was stopped with 50 μl H₂SO₄ 2M and absorbance was read at 450 nm. Plates were washed five times with washing buffer (PBS containing 0.1% (v/v) Tween 20) after each step.

### Shielded Nanoparticle-Asparaginase Protection to Pronase:

Free asparaginase, partially shielded asparaginase or shielded nanoparticle-asparaginase (15-g/ml) were treated with 10 μg/ml of Pronase and incubated in phosphate buffer (0.5M, pH 7.4) at 37° C. under shaking at 300 rpm for different times (1 to 19 hours). After incubation, the enzymatic activity was measured as previously described. “Partially shielded asparaginase” corresponds to a nanoparticle produced as described in WO2015/014888 A1, wherein around 25% of the enzyme asparaginase is embedded by the protective layer. “Shielded nanoparticle-asparaginase” corresponds to a nanoparticle produced as described in WO2015/014888 A1, wherein the enzyme asparaginase is fully embedded by the protective layer.

### Cell Culture:

Human hepatocarcinoma cell line, HepG2, was obtained from CLS (300198). Cells were cultured at 37° C. and 5% CO2 in RPMI 1640 supplemented with 10% heat-inactivated fetal calf serum, 2 mM L-glutamine and 100 U/mL Penicillin/Streptomycin. All media and reagents were purchased from Thermo Scientific. PANC-1 (human pancreatic carcinoma cell line) and MDA-MB-231 (human breast cancer cell line) cells were cultured in DMEM supplemented with 10% heat-inactivated fetal calf serum, 2 mM L-glutamine, 1% non-essential amino-acid and 100 U/mL penicillin/streptomycin. JHH5 (human hepatocellular carcinoma cell line) cells were cultured in RPMI supplemented with 10% heat-inactivated fetal calf serum, 2 mM L-glutamine, 1% non-essential amino-acid and 100 U/mL penicillin/streptomycin. RAJI (human Burkitt lymphoma cell line) cells were cultured in RPMI supplemented with 10% heat-inactivated fetal calf serum, 2 mM L-glutamine, 1% non-essential amino-acid and 100 U/mL penicillin/streptomycin.

### Cytotoxicity Assay:

HepG2 cell line was plated into 96-well flat bottom cell culture plate at a density of 2×10⁴ cells/well. After 24 h, culture medium was replaced, and cells were treated with increasing concentrations of nanoparticles (0-1000 μg/mL), or with digitonin (30 μg/mL) for 48 h. Replicates of nanoparticles without cells were used as blank.

LDH activity assay was performed using a commercial kit. Reagents were prepared according to manufacturer's instructions. Positive controls were treated with 0.1% Triton-X-100 and incubated at room temperature for 10 min. The plate was then centrifuged for 5 min at 450 g. Fifty microliters of cells supernatant were transferred into assay plate and mixed with equal volume of mixed detection kit reagents. After 20 min of incubation, the absorbance was measured at 490 nm using a Synergy H1 Multimode Microplate Reader (Bio-Tek Instruments).

Finally, the LDH leakage was expressed as a percentage of cytotoxicity [(samples absorbance−cell free sample blank)/(Triton-X positive control absorbance−media control absorbance)].

### Hemolysis Assay:

Human blood samples were freshly obtained from healthy adult volunteers at the blood donation center of Basel (Blutspendezentrum SRK beider Basel). Blood was collected in vacutainer tubes containing lithium-heparin as anticoagulant. For hemolysis measurement, diluted whole blood (contain 10±2 mg/mL total blood hemoglobin) was exposed to increasing concentrations of nanoparticles (0 to 1000 μg/mL) for 3 hours at 37° C., mixing the tubes every 30 minutes. Triton (10 mg/mL) and PBS were used as positive and negative controls, respectively. After exposure, the blood suspensions were centrifuged (800 g, 15 min) and CHR reagent (Drabkin's solution containing cyanide) was added to detect the stable form of hemoglobin in the supernatant spectrophotometrically at 540 nm using a Synergy H1 Multimode Microplate Reader (Bio-Tek Instruments).

The amount of released hemoglobin upon hemolysis was analyzed using a calibration curve of Drabkin's reagent and quality controls from commercially available human hemoglobin.

### Biodistribution:

Sprague Dawley rats or CD-1 mice were injected intravenously in lateral tail vein or retro-orbitally with 50 mg/kg of 177Lu-DOTA-Nanoparticles. At terminal time points, the animals were anesthetized by intraperitoneal injection of a mixture of ketamine hydrochloride (50 mg/kg) and xylazine hydrochloride (10 mg/kg), and then they were rapidly sacrificed by exsanguination via intracardiac puncture.

The organs of interest were excised, rinsed in physiological serum and weighed using a precision balance. The counting of tissue radioactivity was performed in an automatic gamma counter (Wallace Wizard 2470—Perkin Elmer) calibrated for Lutetium-177 radionuclide (efficiency: 13.8%; LLOQ: 500 cpm). The radioactivity in sampled tissues was expressed as percentage of the ID per gram of tissue (% ID/g).

Anti-Tumor Efficacy Assay: Cells were plated into 96-well flat bottom cell culture plate at a density of 5×10⁴ cells/well. After 24 h, culture medium was replaced, and cells were treated with increasing concentrations of free asparaginase, NP-1, inactivated NP-1, NP-2, inactivated NP-2 or NP-5 (0-2000 mU/mL) for 48 h. Cellular monolayers were rinsed with medium, and MTT (3-[4,5-dimethylthiazol-2-yl]-2,5-diphenyl tetrazolium bromide) solution (1 mg/mL) was added to each well. The cell cultures were incubated at 37° C. for 2 h. The formazan crystals generated during the incubation period were dissolved in DMSO. After complete solubilization, the absorbance was measured at 570 nm and 680 nm (reference wavelength) using a Synergy H1 Multimode Microplate Reader (Bio-Tek Instruments). Finally, the MTT results were expressed as a percentage of viability: [(samples absorbance−cell free sample blank)×100/(untreated samples absorbance−cell free sample blank)]. Viability of non-treated control cells was arbitrarily defined as 100%.

### Proliferation Assay on PBMCs (Peripheral Blood Mononuclear Cells):

Following separation by Ficoll gradient, PBMCs from healthy donors were immediately labeled with 1 μM CFSE for 10 min at 37° C. and washed with PBS, according to the manufacturer's instructions. Labeled cells were cultivated in complete medium with increasing concentrations of NP-1 or NP-2 (0 to 1000 ug/mL) or with PHA (10 ug/mL) as positive control for 72 h. In all experiments, CFSE dilution was analyzed by flow cytometry.

### Cancer Cells Targeting:

Fluorescent NP-1 and fluorescent NP-5 (50 μg) were incubated with RAJI cells for 30 min in PBS-FBS 1% on ice. After washing and resuspension in PBS, the binding of nanoparticles on RAJI cells were evaluated by flow cytometry and analyzed using the software FlowJo.

### Results:

### Example 1: PEG Functionalization

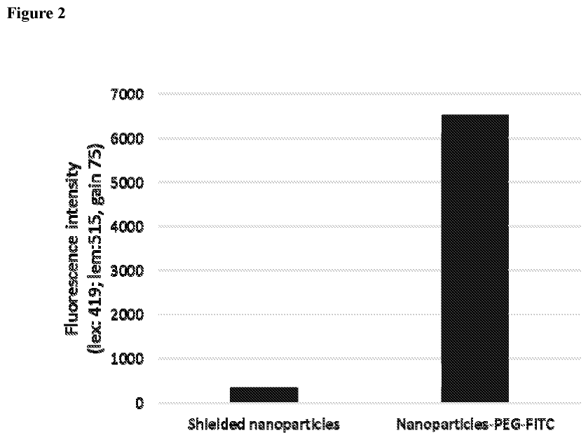

To evaluate the ability to engraft PEG at the surface of shielded nanoparticles (nanoparticles 1, NP-1), a silane-PEG-FITC (NP-2) has been used. The increase of fluorescence after the reaction of polycondensation on reacted nanoparticles (nanoparticles-PEG-FITC, NP-2) compared to shielded nanoparticles NP-1 (6518 vs 343 respectively) as displayed in FIG. 2 demonstrates the immobilization of PEG-FITC at the surface of nanoparticles. This result validates the strategy to functionalize nanoparticles with PEG.

### Example 2: Albumin Functionalization

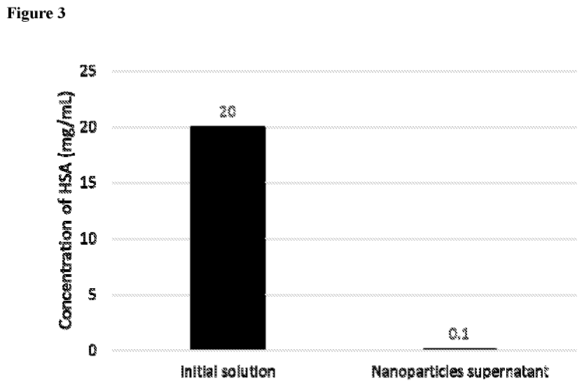

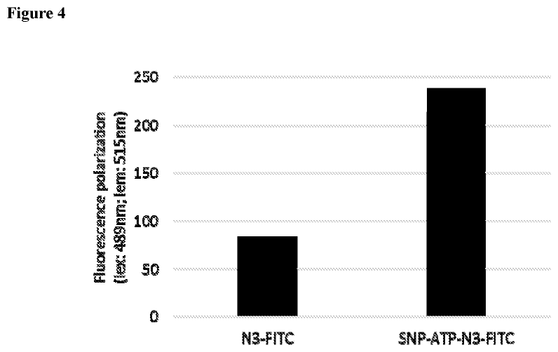

The surface functionalization of shielded nanoparticles (nanoparticles 1, NP-1) with HSA was evaluated by a protein quantification in the supernatant of nanoparticles after reaction with HSA (Nanoparticles 3). The protein concentration in the supernatant of nanoparticles drastically decreased compared to the initial solution of HSA (0.1 vs 20 μg/mL respectively) as displayed in FIG. 3 showing the immobilization of HSA at the surface of the nanoparticles. This result shows the ability to functionalized shielded nanoparticles with proteins.

### Example 3: Click Chemistry Functionalization

To assess the surface functionalization of shielded nanoparticles (nanoparticles 1, NP-1) using the click chemistry, ethynyl-modified nanoparticles were reacted with azido-FITC (N₃—FITC) to obtain Nanoparticles 4 The comparison of fluorescence intensity of free N₃-FITC and nanoparticles after reaction as displayed in FIG. 4 shows an increase of the fluorescence polarization (84 vs 238 respectively). This result demonstrates the immobilization of N₃-FITC at the surface of the nanoparticles by click-chemistry and validate this process to functionalize nanoparticles with further azido-modified compounds.

### Example 4: Asparaginase Activity Assay: Stability of Nanoparticles Produced as Described in WO2015/014888 A1 Compared to Free Enzyme

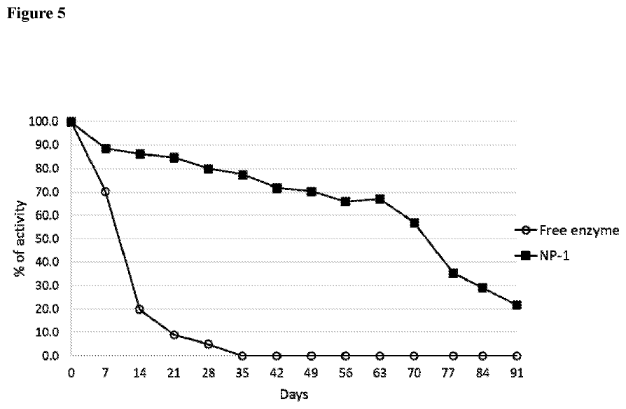

The stability of the biocatalytic activity on shielded nanoparticles (nanoparticles 1, NP-1) was assessed using the Nessler's reaction and compared to the free enzyme over a period of 13 weeks at 37° C. The results as displayed in FIG. 5 show an increase of the half-life of the enzymatic activity on NP-1 compared to the free enzyme (74 days vs 10 days respectively), highlighting the benefits of the protective layer of organosilane on the stabilization of the biocatalytic activity on NP-1.

### Example 5: Asparaginase Activity Assay of Nanoparticles Produced as Described in WO2015/014888 A1 in Biological Fluids

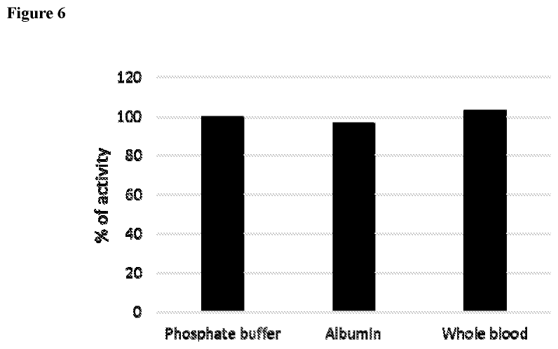

The enzymatic activity of NP-1 in human serum albumin (40 mg/mL) and in whole blood was measured using the Nessler's reaction and compared to the enzymatic activity in phosphate buffer. The results as displayed in FIG. 6 show a sustained enzyme activity of NP-1 in biological fluids.

### Example 6: ELISA: Anti-Asparaginase Antibody Response of Nanoparticles Produced as Described in WO2015/014888 A1

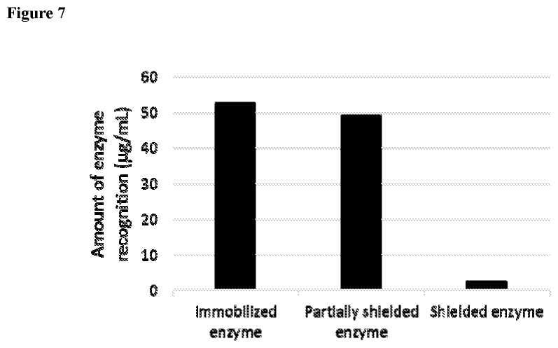

The antibody access to shielded enzymes on the NP-1 was evaluated by ELISA (enzyme-linked immunosorbent assay). Nanoparticles at different stages of their development and the final NP-1 were incubated with an anti-asparaginase antibody. In the case of nanoparticles precursors (immobilized enzymes and partially shielded enzymes), significant binding of anti-asparaginase antibody is observed. However, as displayed in FIG. 7, when the asparaginase is fully shielded (NP-1), the recognition of the enzyme by an anti-asparaginase antibody is drastically reduced. This lack of antibody recognition demonstrates the prevention of antibody access to the enzyme on NP-1.

### Example 7: Asparaginase Activity Assay: Protection to Pronase of Nanoparticles Produced as Described in WO2015/014888 A1

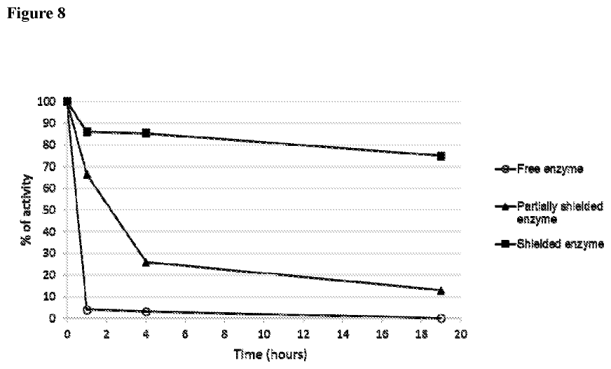

To assess the resistance of NP-1 to external stresses, free enzymes, partially shielded enzymes and fully shielded enzymes (NP-1) were exposed to proteases for 20 h and the remaining enzymatic activity was evaluated using the Nessler's reaction. As expected, after incubation with pronase, no activity was measured for the free asparaginase, and an 85% loss of activity was measured for partially shielded enzyme as displayed in FIG. 8. In the case of nanoparticles 1, a 25% loss of biocatalytic activity was reported, suggesting that the accessibility of the immobilized and shielded enzyme hindered protease digestion.

### Example 8: Cytotoxicity Assay: LDH—48 h of Enzyme-Inactivated Nanoparticles Produced as Described in WO2015/014888 A1

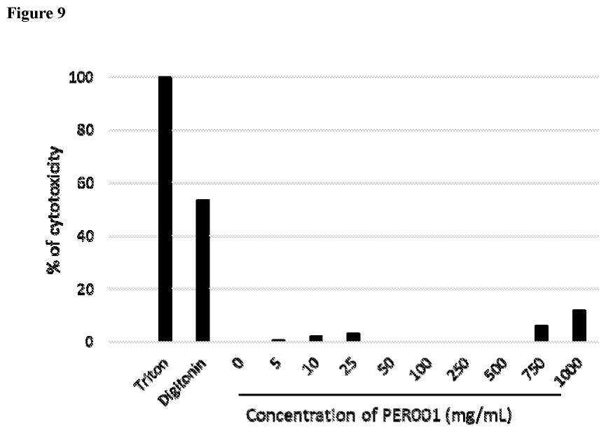

In order to evaluate the biocompatibility of nanoparticles, hepatocarcinoma cells (HepG2) were treated with increasing concentrations of enzyme-inactivated-NP-1 (5 to 1000 μg/mL) for 48 h. Cell damages were assessed by the measurement of the release of lactate deshydrogenase (LDH) in the supernatant. The results as displayed in FIG. 9 show that NP-1 do not impact the viability of HepG2 cells.

### Example 9: Hemolysis Assay of Nanoparticles Produced as Described in WO2015/014888 A1

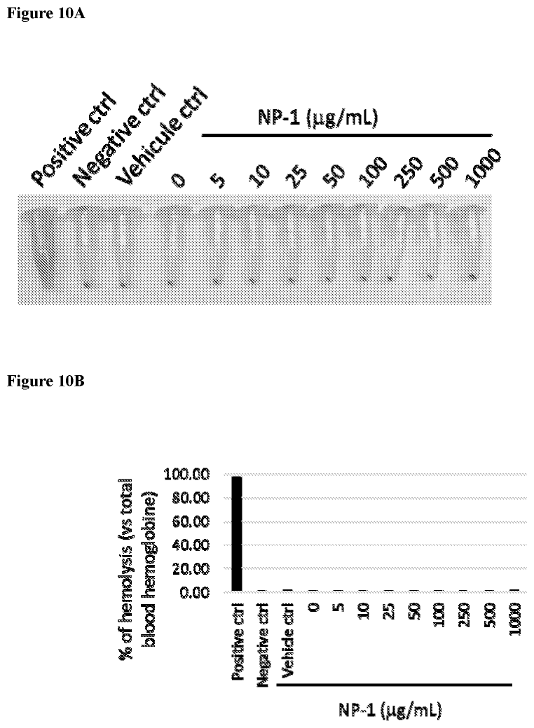

To evaluate the potential hemolytic activity of NP-1, increasing amounts of enzyme-inactivated-NP-1 were added to whole blood for 3 h at 37° C. After incubation, no hemolysis was observed at any conditions as displayed in FIG. 10, pointing out the biocompatibility of NP-1.

### Example 10: Biodistribution (Comparative Experiment)

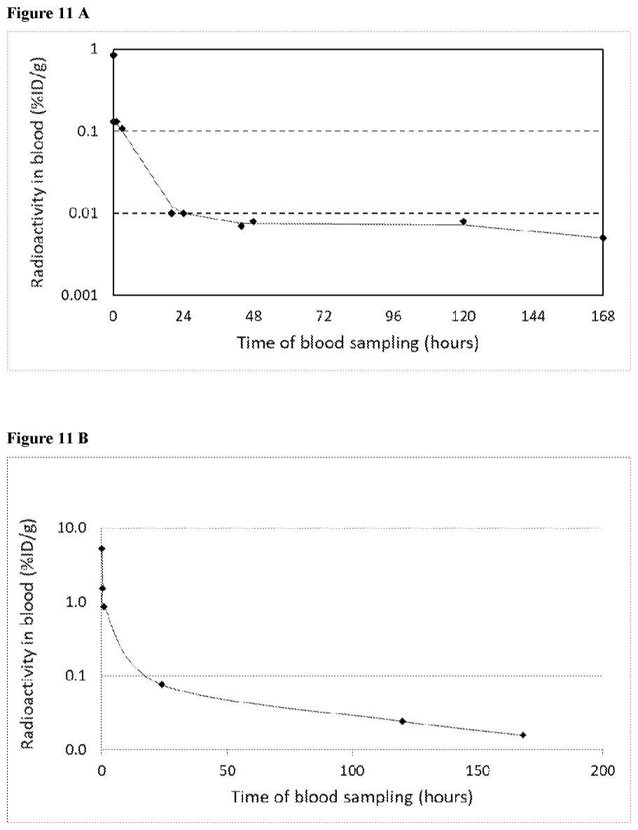

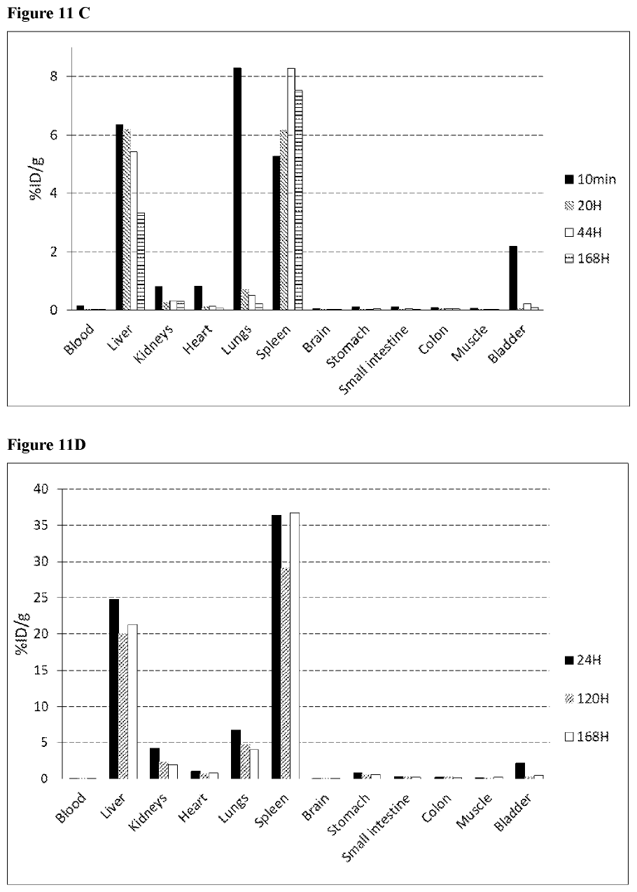

To evaluate the impact of the functionalization of shielded nanoparticles on tissues biodistribution, Lutetium-177-radiolabeled-DOTA (177Lu-DOTA) shielded nanoparticles (NP-1-D) (FIG. 11 A/C) and pegylated shielded nanoparticles (NP-2-D) (FIG. 11 B/D) were injected at 50 mg/kg in animals. Tissues and blood radioactivity were performed at different time points (0 to 7 days). Results show that nanoparticles were quickly cleared from the bloodstream 10 min after injection (FIG. 11 A/B). However, it appears that the amount of pegylated nanoparticles (NP-2-D) is 10 times higher than unmodified nanoparticles (NP-1-D) in the blood circulation. In both cases, the liver and the spleen were the major sites of removal of 177Lu-DOTA-nanoparticles from blood (FIG. 11 C/D). The comparison of the % ID/g of both formulations of nanoparticles shows that the retention of pegylated nanoparticles (NP-2-D) in the organism is higher than NP-1-D. Altogether, these in vivo data reveals that the surface modification of the nanoparticles with PEG allows the modulation of the biological answer and suggest the possibility to control the fate of injected nanoparticles by modulating their surface.

### Example 11: Anti-Tumor Efficacy of NP-2

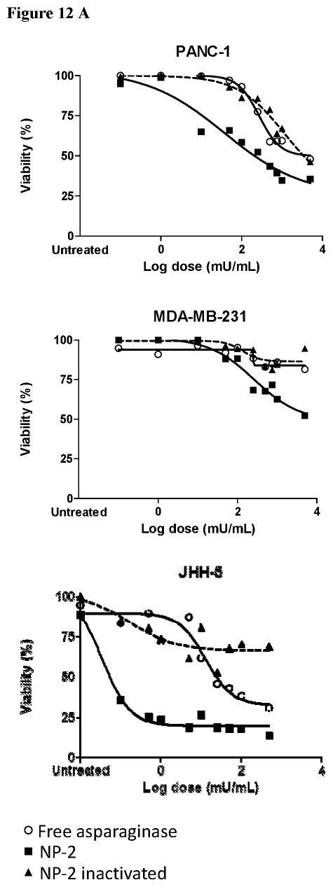

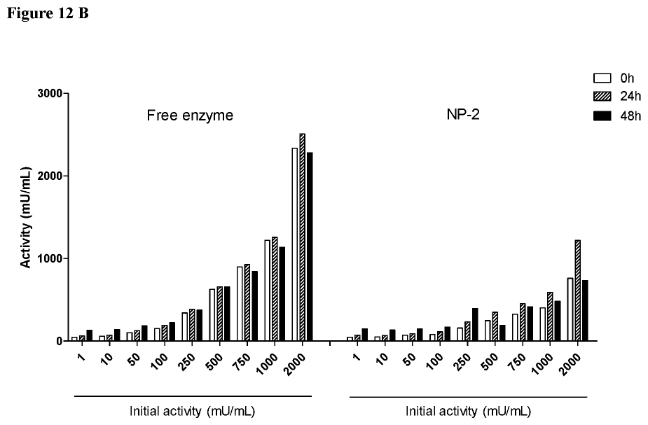

To evaluate the anti-tumor efficacy of NP-2, viability assays were performed on different human cancer cells. Cells were exposed to increasing concentrations of NP-2 or free asparaginase (1 to 2000 mU/mL) or to an equivalent amount of inactivated NP-2 as negative control. FIG. 12A shows a more efficient anti-tumor efficacy of NP-2 compared to free asparaginase in the three cancer cell lines. Asparaginase exerts its anti-tumor efficacy by depleting asparagine in the tumor environment. To explain the differences observed in the anti-tumor effect of NP-2 and free asparaginase, we assessed the enzymatic activity of these two compounds. Surprisingly, results show a higher enzymatic activity of the free enzyme compared to the NP-2 on the range tested (FIG. 12B1), which is inversely correlated to the anti-tumor activity of the compounds. This result shows that the enhanced anti-tumor efficacy of functionalized nanoparticles such as NP-2 seems to be related to a prolonged metabolic restriction induced by the functionalized nanoparticles.

### Example 12: PBMCs Proliferation (Comparative Experiment)

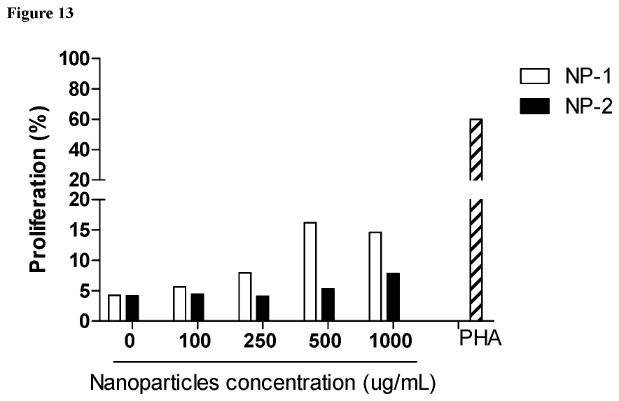

The immune safety of nanoparticles as described in WO2015/014888 (NP-1) and the nanoparticles according to the present invention (NP-2 as described in section “Functionalization with PEG” above) was assessed by measuring the capacity of the nanoparticles to induce human lymphocytes proliferation. Freshy isolated PBMCs were labeled with CFSE and exposed to complete medium without nanoparticles (negative control, untreated condition), increasing concentrations of NP-1 and NP-2 (100 to 1000 ug/mL), or to PHA (positive control) for 72 h. The proliferation of lymphocytes was assessed by flow cytometry. FIG. 13 shows lymphocytes proliferation induced by NP-1 and demonstrates an immune response (lymphocytes proliferation) induced by NP-1 whereas NP-2 do not promote lymphocytes proliferation (compared to the untreated condition). This result demonstrates surprising significant benefits of the nanoparticles of the present invention compared to the nanoparticles as described in WO2015/014888 in terms of immune safety.

### Example 13: Anti-Tumor Efficacy of NP-5 (Comparative Experiment)

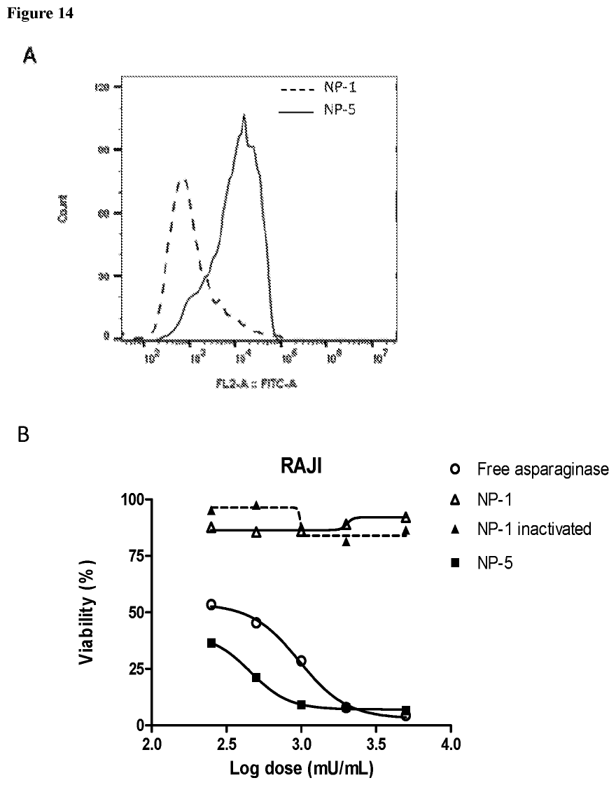

To evaluate the specific targeting of cancer cells by functionalized nanoparticles and their therapeutic applications, anti-CD19 antibodies were immobilized at the surface of the nanoparticles. After co-incubation of fluorescent NP-5 with RAJI cells, their binding on cancer cells were assessed by flow cytometry. Results show that the functionalization of the nanoparticles with an anti-CD19 antibody induces a targeting of cancer cells whereas the unmodified nanoparticles (NP-1) do not interact with the cells (FIG. 14A). The therapeutic impact of this interaction was evaluated by a viability assay. RAJI cells were exposed to increasing concentrations of NP-5, NP-1 or inactivated NP-1(200 to 2000 mU/mL) for 15 min at 37° C., and then unbound nanoparticles were removed by washing. The viability assays were performed for 48 h with free asparaginase as positive control. FIG. 14 B shows an anti-tumor effect of NP-5 whereas the non-functionalized nanoparticles (NP-1) do not trigger any cell toxicity, which was unexpected as NP-1 carries active asparaginase which should have an effect on cell viability in this assay. Moreover, NP-5 shows a more efficient anti-tumor efficacy on RAJI cells compared to the free asparaginase. Altogether, these results demonstrate that the specific targeting of cancer cells promoted by the surface functionalization of the nanoparticles allows a strong interaction between the cancer cells and the nanoparticles, leading to a surprisingly enhanced anti-tumor efficacy.

1. A composition comprising a solid carrier, an enzyme or a fragment thereof immobilized on the surface of the solid carrier, a protective layer to protect the enzyme or the fragment thereof by embedding the enzyme or the fragment thereof, and a functional constituent immobilized on the surface of the protective layer.

2. The composition according to claim 1, wherein the functional constituent immobilized on the surface of the protective layer i) reduces phagocytosis of the composition; ii) increases the circulation time of the composition; and/or iii) targets a tumor and/or promotes the internalization of the composition into tumor cells, when the composition is administered to a subject.

3. The composition according to claim 1 or 2, wherein the functional constituent immobilized on the surface of the protective layer is selected from the group consisting of an amphiphilic drug, an amino acid, a peptide, a protein or a fragment thereof, a silane copolymer, and a combination of a protein or a fragment thereof and a silane copolymer, with the proviso that the protein or the fragment thereof is not the enzyme or the fragment thereof immobilized on the surface of the solid carrier.

4. The composition according to anyone of claims 1-3, wherein the functional constituent is immobilized on the surface of the protective layer by covalent binding.

5. The composition according to claim 1, wherein the functional constituent is selected from the group consisting of serum albumin or a fragment thereof, serum albumin or a fragment thereof and a polyethylene glycol/silane copolymer; a polyethylene glycol/silane copolymer; a Fc fragment of an immunoglobulin; and a Fc fragment of an immunoglobulin and a cross-linker.

6. The composition according to claim 1, wherein the functional constituent is a polyethylene glycol/silane copolymer, preferably a mSilane-PEG 2 kDa or a mSilane-PEG 5 kDa.

7. The composition according to claim 1, wherein the functional constituent is serum albumin or a fragment thereof.

8. The composition according to claim 1, wherein the functional constituent is selected from the group consisting of a peptide; a peptide and a cross-linker; an immunoglobulin or a fragment thereof, and an immunoglobulin or a fragment thereof and a cross-linker.

9. The composition according to anyone of claims 1-8, wherein the enzyme or a fragment thereof is a hydrolase or a fragment thereof.

10. The composition according to anyone of claims 1-8, wherein the enzyme or a fragment thereof is an aspariginase or a fragment thereof.

11. The composition of anyone of claims 1-10, wherein the protective layer embeds the solid carrier and embeds the enzyme or a fragment thereof immobilized on the surface of the solid carrier.

12. The composition of anyone of claims 1-11, wherein the functional constituent immobilized on the surface of the protective layer is not embedded by the protective layer.

13. The composition according to anyone of claims 1-12, wherein the composition further comprises a chelating agent, wherein the chelating agent optionally comprises a radioactive or luminescent label.

14. The composition of anyone of claims 1-13, for use as a medicament.

15. The composition of anyone of claims 1-13, for use in a method for the prevention, delay of progression or treatment of cancer in a subject, the method comprising administering to the subject said composition, wherein the composition is administered in an amount that is sufficient to treat the subject.

16. The composition of claim 13, for use in a method for measuring the distribution of the composition in a subject.

17. A method of producing a composition, the composition comprising a solid carrier, an enzyme or a fragment thereof immobilized on the surface of the solid carrier, a protective layer to protect the enzyme or the fragment thereof by embedding the enzyme or the fragment thereof, and a functional constituent immobilized on the surface of the protective layer, the method comprising the following steps:
(a) providing a solid carrier;
(b) immobilizing an enzyme or a fragment thereof on the solid carrier;
(c) forming a protective layer on the surface of the solid carrier to protect the enzyme or the fragment thereof immobilized on the solid carrier;
(d) immobilizing a functional constituent on the surface of the protective layer.
